(19) United States
(12) Patent Application Publication
(10) Pub. No.: US 2023/0256011 A1
(43) Pub. Date: Aug. 17, 2023

(54) Chlorite In the Treatment of Acute Respiratory Distress Syndrome

(71) Applicant: The Regents of the University of California, Oakland, CA (US)

(72) Inventor: Michael S. McGrath, Burlingame, CA (US)

(21) Appl. No.: 18/002,385

(22) PCT Filed: Jun. 24, 2021
(86) PCT No.: PCT/US2021/038959

§ 371 Date: Dec. 19, 2022

Related U.S. Application Data
(60) Provisional application No. 63/046,215, filed on Jun. 30, 2020.

(51) Int. Cl. A61K 33/20; A61P 31/14; A61K 9/00

(52) U.S. Cl. CPC A61K 33/20; A61K 9/0019; A61P 31/14

(57) ABSTRACT

Disclosed herein are methods of treating a subject having, or at risk of developing, acute respiratory distress syndrome (ARDS) by administering a chlorite composition in an amount effective to treat the subject. Aspects of the methods also include administering a chlorite formulation to a subject having or suspected of having a coronavirus infection, e.g., severe acute respiratory syndrome coronavirus 2 (SARS-CoV-2), severe acute respiratory syndrome (SARS-CoV), or Middle East respiratory syndrome coronavirus (MERS-CoV) infection. Also disclosed herein are methods of regulating macrophage activity in a subject having, or at risk of developing ARDS by contacting a macrophage of a subject having or at risk of developing ARDS with a chlorite composition in an amount effective to regulate one or more functional properties of the macrophage. The disclosure also features methods of determining inhibition of a ARDS-induced macrophage activity by a chlorite agent.

### CROSS-REFERENCE TO RELATED APPLICATIONS

This application claims priority benefit to U.S. Provisional Application Serial No. 63/046,215, filed Jun. 30, 2020, which application is incorporated by reference in its entirety.

### FIELD OF THE DISCLOSURE

The present disclosure generally relates to the use of a chlorite formulation in treatment of acute respiratory distress syndrome, particularly respiratory distress syndrome caused by an infectious pathogen. The present disclosure also relates to the use of a chlorite formulation for the treatment of a coronavirus infection.

### INTRODUCTION

In recent years there have been life-threatening respiratory conditions caused by new strains of coronaviruses such as those found in the 2003 severe respiratory syndrome (SARS) outbreak, Middle East Respiratory Syndrome (MERS) in 2012, and most recently the 2019 coronavirus outbreak originating from Wuhan, China (SARS-CoV-2). Human infection with SARS-Cov-2 can lead to two general clinical outcomes based on the pattern of disease pathogenesis. The first is a relatively severe conventional upper airway epithelial cell viral infection with normal immune system clearance within 1-2 weeks; the second involves chronic infection of tissue based lung and other organ macrophages, a process that leads to a self-perpetuating “cytokine storm” wherein tissue macrophages elaborate factors that lead to persistence of a type of destructive tissue based inflammation. In some cases, patients with the second and most severe outcome require intensive care and respiratory support, and many of the patients requiring intensive care have developed acute respiratory distress syndrome (ARDS). Acute respiratory distress syndrome (ARDS) is a severe lung inflammation condition that leads to fluid build up in the lung alveoli. The fluid prevents the lungs from filling with enough air, limiting the amount of oxygen that reaches the bloodstream, and thus deprives the bodie’s organs of oxygen.

There is currently a need for therapeutic options for treating ARDS, particularly ARDS that arises as a result of viral infection such as SARS-CoV-2 infection.

### SUMMARY

Disclosed herein are methods of treating a subject having, or at risk of developing, acute respiratory distress syndrome (ARDS) by administering a chlorite composition in an amount effective to treat the subject. Aspects of the methods also include administering a chlorite formulation to a subject having or suspected of having a coronavirus infection, e.g., severe acute respiratory syndrome coronavirus 2 (SARS-CoV-2), severe acute respiratory syndrome (SARS-CoV), or Middle East respiratory syndrome coronavirus (MERS-CoV) infection. Also disclosed herein are methods of regulating macrophage activity in a subject having, or at risk of developing, ARDS by contacting a macrophage of a subject having or at risk of developing ARDS with a chlorite composition in an amount effective to regulate one or more functional properties of the macrophage. The disclosure also features methods of determining inhibition of an ARDS-induced macrophage activity by a chlorite agent.

### BRIEF DESCRIPTION OF THE DRAWINGS

The invention is best understood from the following detailed description when read in conjunction with the accompanying drawings. Included in the drawings are the following figures:

FIG. 1, panels A-D illustrates that plasma levels of factors associated with COVID-19 “cytokine storm” that are significantly elevated in plasma of patients as compared to respective controls NS: non-significant. P<0.05 (*); p<0.005 (**); p<0.0001 (***). (Blanco-Melo et al. 2020, Cell, 181:5, 1036-1045).

FIG. 2 illustrates that a sodium chlorite formulation regulates in vitro CD16 expression on ALS macrophages, providing evidence for dose-dependent blockage of MO activation.

FIG. 3 illustrates the effect of a sodium chlorite formulation on inflammatory gene expression in ALS patient macrophages.

FIG. 4 illustrates that a sodium chlorite formulation inhibits production of secreted inflammatory factors from ALS patient macrophages, showing the effect of the sodium chlorite formulation on various inflammatory protein secretions.

FIG. 5 depicts COVID-19 lower lung disease macrophages in severe COVID-19 disease: M2 like MOs (red) infected with SARS-Cov-2 (brown).

FIG. 6, panels A-B illustrates that a sodium chlorite formulation inhibits production of monocyte associated osteopontin (OPN) from ALS patient macrophages (panel A); and that a sodium chlorite formulation inhibits SPP1 (osteopontin) gene expression in a dose-dependent manner. Note: RT-PCR for OPN RNA shows same level of regulation. Repeated with 3 normal and 5 ALS donors (panel B).

FIG. 7, panels A-B depicts pie charts from microbiome profiling of two groups of COVID 19 patients, namely alive/discharged patients (panel A) vs deceased patients (panel B). Each pie chart shows the relative abundance of different bacterial families, notably two bacterial families differ between the groups indicated (Flavobacteriaceae and Propionibacteriaceae).

FIG. 8, illustrates that a sodium chlorite formulation downregulated production of COVID-19 patient macrophage production disease factors as compared to controls, showing the effect of the sodium chlorite formulation on various inflammatory protein secretions.

### DETAILED DESCRIPTION

As outlined above, disclosed herein are methods of treating a subject having, or at risk of developing, acute respiratory distress syndrome (ARDS) by administering a chlorite composition in an amount effective to treat the subject. Aspects of the methods also include administering a chlorite formulation to a subject having or suspected of having a coronavirus infection, e.g., severe acute respiratory syndrome coronavirus 2 (SARS-CoV-2), severe acute respiratory syndrome (SARS-CoV), or Middle East respiratory syndrome coronavirus (MERS-CoV) infection. Also disclosed herein are methods of regulating macrophage activity in a subject having, or at risk of developing, ARDS by contacting a macrophage of a subject having or at risk of developing ARDS with a chlorite composition in an amount effective to regulate one or more functional properties of the macrophage. The disclosure also features methods of determining inhibition of an ARDS-induced macrophage activity by a chlorite agent.

Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

While the apparatus and method has or will be described for the sake of grammatical fluidity with functional explanations, it is to be expressly understood that the claims, unless expressly formulated under 35 U.S.C. §112, are not to be construed as necessarily limited in any way by the construction of “means” or “steps” limitations, but are to be accorded the full scope of the meaning and equivalents of the definition provided by the claims under the judicial doctrine of equivalents, and in the case where the claims are expressly formulated under 35 U.S.C. §112 are to be accorded full statutory equivalents under 35 U.S.C. §112.

### Definitions

The term “acute respiratory distress syndrome” or “ARDS” refers to a syndrome characterized by a severe shortness of breath, labored and unusually rapid breathing, low blood pressure, confusion and extreme tiredness. ARDS occurs when fluid builds up in lung alveoli. The fluid prevents the lungs from filling with enough air, limiting the amount of oxygen that reaches the bloodstream which, in turn, deprives the organs of the oxygen they need to function. The symptoms of ARDS can vary in intensity, depending on its cause and severity. In some cases, ARDS is caused by a microbial pathogen. In some cases, ARDS is caused by a respiratory virus, e.g., a coronavirus, or an influenza virus.

As used herein, the terms “macrophage” and “monocyte” are used interchangeably, as it is understood that in the art the term “monocyte” is often used to describe a circulating mononuclear cell that expresses the CD14 cell surface marker, and when in a tissue this cell is also classified as a macrophage.

An “abnormal macrophage” or “activated circulating monocyte” or “activated monocyte” as used interchangeably herein denotes a monocyte which expresses CD14 (i.e., CD14+) and which expresses an elevated level of HLA-DR, the maj or histocompatibility antigen class II, and/or which expresses CD16 (i.e., CD16+). Generally, abnormal macrophages are found in peripheral blood but they may also be found in other biological samples from an individual..

As used herein, detecting the “presence of abnormal macrophages” generally means detecting the level of abnormal macrophages within blood or a biological fluid. Generally, the level of abnormal macrophages (or activated monocytes) is indicated by the level of HLA-DR expression in a population of CD14+ cells and/or the percentage of CD16+ cells in a population of CD14+ cells and/or the number of CD14+/CD16+ cells, although other markers that indicate monocyte activation, differentiation and/or proliferation could be used. It is understood that an absolute or even relative level need not be determined; an observation of detectable abnormal macrophages is sufficient.

“Pathologic macrophages” as used herein is meant to encompass inappropriately activated macrophages (e.g., abnormal macrophages). Pathologic macrophages are nonetheless chronically activated, and thus are in a pathogenic state.

A “macrophage-associated” disease, disorder or indication is a disease, disorder or indication that is associated with pathologic macrophages an elevated, or abnormal, level or rate of macrophage activation as compared to control sample(s). Such disorders include, but are not limited to, macrophage-associated respiratory disorders, such as ARDS as observed in COVID-19. The terms “disorder” and “disease” are used interchangeably herein. An “ARDS-associated” disease is defined more broadly as generally associated with or secondary to a pathogen infection, usually a respiratory pathogen infection; “ARDS-mediated” diseases, for example, are included in those considered to be “ARDS-associated.” A “coronavirus-associated” disease is defined more broadly as generally associated with or secondary to a coronavirus infection; “coronavirus-mediated” diseases, for example, are included in those considered to be “coronavirus-associated.” In particular embodiments, the disorder contemplated for treatment according to the invention is not cancer. In other particular embodiments, the disorder contemplated for treatment according to the invention is not an autoimmune disease. For example, the disorder is not graft rejection (transplant rejection), is not a neurological disease (e.g.,. the disease is not a macrophage-associated neurodegenerative disease (such as amyotrophic lateral sclerosis (ALS), multiple sclerosis (MS) or Alzheimer’s disease (AD)), and/or is not a disorder caused by or associated with HIV infection.

A “macrophage-associated ARDS individual” or a “macrophage-associated ARDS patient” is an individual who is diagnosed as having ARDS or is suspected of having ARDS by demonstrating clinical symptoms of ARDS, which symptoms include pathologic macrophages in the patient’s blood. A “non-macrophage-associated ARDS individual” is an individual who is not diagnosed as having, and not suspected of having, a macrophage-associated ARDS.

An “ARDS individual” or an “ARDS patient” is an individual who is diagnosed as having ARDS or is suspected of having ARDS by demonstrating ARDS-associated symptoms. A “non-ARDS individual” is an individual who is not diagnosed as having ARDS or not suspected of having ARDS. ARDS and methods of diagnosing ARDS are known in the art and are discussed herein.

A “COVID-19 individual” or a “COVID-19 patient” is an individual who is diagnosed as having COVID-19 or is suspected of having COVID-19 by demonstrating COVID-19-associated symptoms. A “non-COVID-19 individual” is an individual who is not diagnosed as having COVID-19 or not suspected of having COVID-19. COVID-19 and methods of diagnosing COVID-19 are known in the art and are discussed herein.

“Development” or “progression” of a disease, e.g., a macrophage-associated respiratory disease such as ARDS, e.g., as can be associated with COVID-19, herein means initial manifestations and/or ensuing progression of the disorder. For example, development of ARDS can be detectable and assessed using standard clinical techniques, such as assays, physical examination, oxygen levels and scanning technologies such as chest X-ray. However, development also refers to disease progression that may be undetectable. For purposes of this disclosure, development or progression refers to the biological course of the disease state. “Development” includes occurrence, recurrence, and onset. As used herein “onset” or “occurrence” of ARDS, e.g., as in COVID-19, includes initial onset and/or recurrence.

As used herein, “delaying development” of a macrophage-associated respiratory disease, such as ARDS, e.g., as in COVID-19, means to defer, hinder, slow, retard, stabilize, and/or postpone development of one or more symptoms, of the disease, including decreasing the rate at which the patient’s disease progresses (e.g., to shift the patient from rapidly progressing disease to a more slowly progressing disease). This delay can be of varying lengths of time, depending on the history of the disorder and/or the medical profile of the individual being treated. As is evident to one skilled in the art, a sufficient or significant delay can, in effect, encompass prevention, in that the individual does not develop detectable disease. A method that “delays” development of disease is a method that reduces the extent of the disease in a given time frame, when compared to not using the method. Such comparisons are typically based on clinical studies, using a statistically significant number of subjects, although this knowledge can be based upon anecdotal evidence. “Delaying development” can mean that the extent and/or undesirable clinical manifestations are lessened and/or time course of the progression is slowed or lengthened, as compared to not administering the agent. Thus the term also includes, but is not limited to, alleviation of symptoms, diminishment of extent of disease, stabilized (i.e., not worsening) state of disease, delay or slowing of disease progression, and remission (whether partial or total) whether detectable or undetectable.

As used herein, “biological sample” encompasses a variety of sample types obtained from an individual and can be used in a diagnostic or monitoring assay. The definition encompasses blood and other liquid samples of biological origin, solid tissue samples such as a biopsy specimen or tissue cultures or cells derived therefrom, and the progeny thereof. The definition also includes samples that have been manipulated in any way after their procurement, such as by treatment with reagents, solubilization, or enrichment for certain components, such as proteins or polynucleotides. The term “biological sample” encompasses a clinical sample, and also includes cells in culture, cell supernatants, cell lysates, serum, plasma, biological fluid, and tissue samples. Generally, the sample will be, or be derived from, peripheral blood and as such is a “blood sample”. In some cases, the blood will have been enriched for a macrophage fraction, by using, for example, glass or plastic adherence.

A “blood sample” is a biological sample which is derived from blood, such as peripheral (or circulating) blood. A blood sample may be, for example, whole blood, plasma or serum.

As used herein, an “effective amount” (e.g., of an agent) is an amount (of the agent) that produces a desired and/or beneficial result. An effective amount can be administered in one or more administrations. In general, an effective amount in the context of the methods of the present disclosure refers to is an amount sufficient to decrease inflammation, particularly respiratory inflammation, in a subject receiving chlorite therapy, where a decrease in inflammation may be assessed by any conventional methods (e.g., by assessing pulmonary function and/or by imaging (e.g., X-ray, ultrasound, MRI), and/or by assessing a level of one or more blood inflammatory markers (e.g., by detecting a decrease in one or more of interleukin-18 (IL-18), C-X-C motif chemokine ligand 9 (CXCL9), C-X-C motif chemokine ligand 10 (CXCL10), interleukin-1 receptor antagonist (IL1Ra), or a combination thereof) and/or by assessing a blood level of abnormal macrophages (pathologic macrophages) in an ARDS patient, e.g., a COVID-19 patient. An “amount sufficient to decrease the level of one or more inflammatory markers” generally refers to a decrease at least about 25%, at least about 50%, at least about 75%, or at least about 90% as compared to a pre-treatment inflammatory marker level. An “amount sufficient to decrease the level of abnormal macrophages” is able to decrease the level of abnormal macrophages by at least about 25%, at least about 50%, at least about 75%, or at least about 90%. Such a decrease may have desirable concomitant effects, such as to palliate, ameliorate, stabilize, reverse, slow or delay progression of disease, delay and/or even prevent onset of advanced disease.

As used herein, decreasing the “level of abnormal macrophages” generally means decreasing the population number of abnormal macrophages or activated monocytes and/or decreasing the level of CD16 expression in a population of CD14+ cells. In various embodiments, the level of abnormal macrophages can be assayed by determining the percentage of CD16+ cells in a population of CD14+ cells and/or the number of CD14+/CD16+ cells in the biological sample. It is understood that an absolute level need not be determined; an observation of a relative level of abnormal macrophages is sufficient.

“Treatment” or “treating” as used herein means any therapeutic intervention in a subject, usually a mammalian subject, generally a human subject, including: (i) prevention, that is, causing overt clinical symptoms not to develop, e.g., preventing disease progression to a harmful state; (ii) inhibition, that is, arresting the development or further development of clinical symptoms, e.g., mitigating existing clinical symptoms; and/or (iii) relief, that is, causing the regression of clinical symptoms, e.g., causing relief from clinical symptoms.

Examples of clinical symptoms of ARDS include severe shortness of breath, labored and unusually rapid breathing, low blood pressure, confusion and extreme tiredness. ARDS can be diagnosed based on a PaO2/FiO2 ratio of less than 300 mmHg despite a PEEP of more than 5 cm H2O (Fan et al., JAMA. 2018, 319:7, 698-710). “Treating” thus encompasses achieving a decrease in one or more clinical symptoms, which decrease may have desirable concomitant effects, such as to palliate, ameliorate, stabilize, reverse, slow or delay progression of disease, delay and/or even prevent onset of disease.

The terms “subject” “individual” and “patient” mean a member or members of any mammalian or non-mammalian species that may have a need for the pharmaceutical methods, compositions and treatments described herein. Subjects and patients of particular interest are those susceptible to coronavirus infection, e.g., SARS-CoV-2 infection, and/or otherwise at risk of developing ARDS. Humans are subjects of particular interest..

The term “unit dosage form,” as used herein, refers to physically discrete units suitable as unitary dosages for a subject, each unit containing a predetermined quantity of compound of the present invention calculated in an amount sufficient to produce the desired effect in association with a pharmaceutically acceptable excipient (e.g., pharmaceutically acceptable diluent, carrier or vehicle).

A “pharmaceutically acceptable excipient” means an excipient that is useful in preparing a pharmaceutical composition that is generally safe, non-toxic and neither biologically nor otherwise undesirable, and includes an excipient that is acceptable for veterinary use as well as human pharmaceutical use. “A pharmaceutically acceptable excipient” as used in the specification and claims includes both one and more than one such excipient.

The term “pharmaceutically acceptable salt” means a salt which is acceptable for administration to a patient, such as a mammal (salts with counterions having acceptable mammalian safety for a given dosage regime). Such salts can be derived from pharmaceutically acceptable inorganic or organic bases and from pharmaceutically acceptable inorganic or organic acids. “Pharmaceutically acceptable salt” refers to pharmaceutically acceptable salts of a compound, which salts are derived from a variety of organic and inorganic counter ions well known in the art and include, by way of example only, sodium, lithium, potassium, calcium, magnesium, and the like.

### Chlorite Compositions

Chlorite compositions that find use in the present disclosure are described below. In certain cases, the chlorite is in the form of a pharmaceutically acceptable salt (e.g., as described herein). In some embodiments, the pharmaceutically acceptable salt is chosen from lithium, potassium, sodium, calcium, and magnesium salts. In certain cases, the salt is a sodium salt, such that the chlorite is sodium chlorite.

In some embodiments, the chlorite composition is formulated in aqueous solution in which the chlorite is greater than 95% pure. In some cases, the chlorite can be greater than 97%, 98%, 98.5%, 99%, 99.5% or 99.9% pure. In some cases, the chlorite can be at least 95%, 97%, 98%, 98.5, 99%, 99.5% or 99.9% pure. In some cases, the chlorite is greater than 98% pure. In some cases, the chlorite is at least 98% pure. In some cases, the chlorite is greater than 99% pure. As used herein, the “purity” of chlorite in a sample is calculated as the percent weight of chlorite salt to the total weight of the sample. In determining the purity of chlorite in a solution, the weight of the solvent (e.g., water in an aqueous solution) is not included. Purity may be evaluated using ion chromatography and an ion detector, by calibrated integration of the respective peaks; for example, chlorite, chloride, chlorate, phosphate and sulfate in the compound or formulation. For example, chlorite is commercially available as sodium chlorite, technical grade, at a purity of 80% (catalog No. 244155 Sigma-Aldrich).

Alternatively, crystalline sodium chlorite is provided in a purity greater than 95%, greater than 96%, greater than 97%, greater than 98%, greater than 99%, greater than 99.5% or greater than 99.9%. Solid pharmaceutical formulations comprising crystalline sodium chlorite in a purity greater than 95%, greater than 96%, greater than 97%, greater than 98%, greater than 99%, greater than 99.5% or greater than 99.9% in addition to one or more pharmaceutical excipients are also encompassed.

The chlorite compositions for use with the present disclosure can comprise low amounts of chlorate, sulfate or chloride. As used herein, a formulation is “substantially free” of a molecule if the molecule comprises no more than 1 part in 1000 per weight of non-solvent molecules in the formulation. In certain embodiments, the weight ratio of chlorite to chlorate is greater than 100:1.5, greater than 100:0.5, greater than 100:1, greater than 100:0.1, or greater than 100:0.05. In one embodiment, the composition is substantially free of chlorate. In another embodiment, the weight ratio of chlorite to chloride is greater than 100:45.5 or greater than 100:8.5. In one embodiment the composition is substantially free of chloride. In a further embodiment, the weight ratio of chlorite to sulfate is greater than 100:16.4 or greater than 100:1.6. In one embodiment the composition is substantially free of sulfate.

The pH of a chlorite aqueous composition for use with the present disclosure can be adjusted to a pH of 8 or more, such as 8.5 or more, 9 or more, or even more. In some cases, the chlorite formulation has a pH from 7 and about 11.5. In some other cases, the chlorite formulation has a pH from 8 to 9. In some embodiments, the pH of a chlorite formulation is lowered to a range of about 7 and about 11.5, or about 8 to 9 using a pH adjusting compound that does not expose the formulation to high local acidity. In some embodiments, the pH adjusting compound is any one or more of monosodium phosphate, disodium phosphate, or acetic acid.

Methods of formulating a chlorite composition have been described in US Patent Pub. No. 20070145328, filed Dec. 21, 2006 and entitled “Chlorite Formulations, and Methods of Preparation and Use Thereof,” which is incorporated herein by reference in its entirety. Such formulations are suitable for various modes of administration, including but not limited to non-topical, parenteral, systemic, or intravenous administration. In certain cases, the chlorite composition is formulated for intravenous administration.

As described herein, in some cases the chlorite composition is an aqueous formulation. In some embodiments, the chlorite formulation comprises an aqueous solvent, and optionally one or more other solvents for chlorite. In some embodiments, the formulations comprise sodium chlorite and an aqueous solvent for sodium chlorite, and have a pH of about 7 to about 11.5, such as about 8 to about 9.

Solvents or combinations of solvents for use in the chlorite compositions described herein can be determined by a variety of methods known in the art. One non-limiting example includes (1) theoretically estimating solvent solubility parameter value(s) and choosing the one(s) that match with chlorite, using standard equations in the field; and (2) experimentally determining the saturation solubility of chlorite in the solvent(s), and (3) choosing one or more that exhibits the desired solubility, and (4) selecting a solvent or solvents that do not diminish the activity of chlorite, or that do not or only minimally react with chlorite. In some embodiments, the liquid formulations described herein comprise a plurality of solvents.

In some embodiments, the chlorite compositions comprise an aqueous solvent. In some variations, water is the principal solvent in the aqueous formulations. In some variations, water is at least about 50% by volume of the solvent component of an aqueous formulation. In some variations, water is at least 50% by volume of the aqueous formulation. In some variations, water is any of from 50 to 60, from 60 to 70, from 70 to 80, from 80 to 90, from 90 to 99, at least 50, at least 60, at least 70, at least 80, at least 90, or at least 95, about 50, about 60, about 70, about 80, about 90, or about 95 percent by volume of the solvent component. In some variations, water is any of from 50 to 60, from 60 to 70, from 70 to 80, from 80 to 90, from 90 to 99, at least 50, at least 60, at least 70, at least 80, at least 90, or at least 95, percent by volume of the aqueous formulation. In some variations, water is at least 95% by volume of the aqueous formulation. In some variations, water is from 80 to 90% by volume of the aqueous formulation. In some variations, water is from 90 to 99% by volume of the aqueous formulation.

The chlorite compositions may have differing concentration of chlorite. In some embodiments, the concentration of chlorite in the formulations described herein is from 1 µM to 1.5 M. In another embodiments, the concentration of chlorite in the formulations described herein is from any of 1 M to 1.5 M; from 1 µM to 100 mM; from 10 µm to 100 mM; from 0.1 mM to 10 mM; from 0.1 mM to 500 mM; from 0.1 mM to 200 mM; from 1 mM to 100 mM; from 0.1 mM to 5 mM; from 50 mM to 100 mM; from 55 mM to 70 mM; from 60 mM to 65 mM; from 100 mM to 500 mM; from 200 mM to 400 mM; from 300 mM to 700 mM; about 1 mM; about 1.5 mM; about 2 mM; about 2.5 mM; about 3 mM; about 3.5 mM; about 4 mM; about 5 mM; about 10 mM; about 20 mM; about 30 mM; about 40 mM; about 50 mM; about 60 mM; about 62 mM; about 65 mM; about 70 mM; about 80 mM; about 90 mM; about 100 mM; 0.1 mM or more; 1 mM or more; 2 mM or more; 5 mM or more; 10 mM or more; 20 mM or more; 30 mM or more, 40 mM or more; 50 mM or more; 60 mM or more; 70 mM or more; 80 mM or more; 90 mM or more, 100 mM or more. In preferred embodiments, the concentration of chlorite in the formulations described herein is about 60 mM or more.

In some embodiments, the concentration of chlorite in the formulation is diluted to a less concentrated form prior to administration. In some embodiments, a formulation described herein is diluted about, at least 2.5×, 5×, 7.5×, 10×, 20×, 25×, 50×, 100×, 200×, 250×, 300×, 500×, or 1000×. In some embodiments, a formulation described herein is diluted from 2× to 10×, from 10× to 50×, from 50× to 100×, from 100× to 500×, or from 500× to 1000×. In some embodiments, a formulation as described herein is diluted from 2× to 10×. In some embodiments, a formulation as described herein is diluted from 10× to 50×. In some embodiments, a formulation as described herein is diluted about 7.5×. In some embodiments, a formulation as described herein is diluted about 25×. In some embodiments, a formulation as described herein is diluted about 200×. In some embodiments, the chlorite formulation is not diluted prior to administration.

In some embodiments, the concentration of chlorate in the formulations described herein is 0.05% or less, such as 0.04% or less, 0.03% or less, 0.02% or less, or even less. In some embodiments, the concentration of chlorite in the formulation is from 50 mM to 100 mM. In some embodiments, the concentration of chlorate in the formulations described herein is from 55 mM to 75 mM. In some embodiments, the concentration of chlorate in the formulations described herein is from 0.1 mM to 10 mM. In some embodiments, the concentration of chlorate in the formulations described herein is from 1 mM to 5 mM.

In some embodiments, the chlorite composition has a pH of 12.0 or less. In some embodiments, the pH of the composition has a pH of 11.5 or less, 11.0 or less, 10.5 or less, 10.0 or less, 9.5 or less, 9.0 or less, 8.5 or less, or 8.0 or less. In some embodiments, the pH of the formulation is no greater than 11.5. In some embodiments, the pH of the formulation is no greater than 10.5. In some embodiments, the pH of the formulation is no greater than 8.5. In some embodiments, the pH of the formulation is no greater than about 7.5. In some embodiments, the pH of the formulation is from any one or more of 7 to 12; 7 to 11.5; 7 to 10.5; 7 to 10; 7 to 9.5; 7 to 9.0; 7 to 8.5; 7 to 8.0; 7 to 7.5; 7.5 to 8; 7.5 to 8.5; 7 to 8; 8 to 9; 7.0 to 8.5; 8 to 8.5; 8.5 to 9; 7.1 to 7.7; 7.2 to 7.6; 7.3 to 7.4; 7.0; to 7.1; about 7.2, about 7.3; about 7.4; about 7.5; about 7.6; about 7.7; about 7.8; about 7.9; about 8.0; about 8.1; about 8.2; about 8.3; about 8.4; about 8.5; about 8.6; about 8.7; about 8.8, or about 8.9. In some embodiments, the chlorite composition has a pH of from 7.0 to 9.0. In some embodiments, the chlorite composition has a pH of about 7.0 to 8.5. In some embodiments, the chlorite formulation has a pH of 8.0 to about 8.5. In some embodiments, the chlorite formulation has a pH of 8.5 to 9.0. In some embodiments, the chlorite formulation has a pH of from 7.0 to 8.0. In some embodiments, the chlorite formulation has a pH of about 7.4. The chlorite formulation can have a pH that is at a physiological level.

In some embodiments, the chlorite formulations have a pH as described above, and are formulated for any one or more of parenteral, systemic, or intravenous administration. In some embodiments, the chlorite formulations have a pH as described above, and have a percentage chlorite purity as described herein.

In some embodiments, the formulations described herein have a pH as described above, and have a concentration of chlorite as described herein. In some embodiments, the aqueous formulations described herein have a pH from 7 to 11.5, or from 8.0 to 9.0, or from 7.5 and about 8.0, or from 8.1 and about 8 5, or from 8.6 to 9.0, and have a concentration of chlorite between about 1 and about 100 mM. In some embodiments, the aqueous formulations described herein have a pH from 7 to 11 5, or from 8.0 to 9.0, or from 7.5 and about 8.0, or from 8.1 and about 8.5, or from 8.6 to 9.0, and have a concentration of chlorite between about 1 and about 5 mM. In some embodiments, the aqueous formulations described herein have a pH from 7 to 11.5, or from 8.0 to 9.0. or from 7.5 and about 8.0, or from 8.1 and about 8.5, or from 8.6 to 9.0, and have a concentration of chlorite between about 50 and about 80 mM.

In some embodiments, the aqueous formulations described herein have a pH from 7 to 11.5, or from 8.0 to 9.0, or from 7.5 and about 8.0, or from 8.1 and about 8.5, or from 8.6 to 9.0, wherein the pH was adjusted with a pH adjusting agent that is any one or more of a phosphate, or acetic acid

In some embodiments, the formulations described herein are stable with respect to one or more of pH or chlorite degradation over a period of any of at least about 1 day, at least about 2 days, at least about 3 days, at least about 4 days, at least about 5 days, at least about 6 days, at least about 1 week, at least about 2 weeks, at least about 3 weeks, at least about 4 weeks, at least about 5 weeks, at least about 6 weeks, at least about 7 weeks, at least about 8 weeks, at least about 1 month, at least about 2 months, at least about 3 months, at least about 4 months, at least about 5 months, or at least about 6 months In some embodiments, the formulations described herein are stable with respect to one or more of pH or chlorite degradation over a period of any of at least about 1 week In some embodiments, the formulations are stable with respect to one or more of pH or chlorite degradation over a period of any of at least about 1 month. In some embodiments, the formulations described herein are stable with respect to one or more of pH or chlorite degradation at one or more of room temperature, refrigerated conditions, or approximately 4° C. In some embodiments, the formulations described herein are stable with respect to one or more of pH or chlorite degradation under conditions of diminished light or storage in a container that limits the amount of light to which the formulation is subjected. In some embodiments, the formulations described herein are stable with respect to one or more of pH or chlorite degradation when stored in the dark. Examples of stable pH, as used herein, means that the pH of the formulation changes by less than any of about 0.1, about 0.2, about 0.3, about 0.4, about 0.5, about 0.6, about 0.7, about 0.8, about 0.9, or about 1 relative to the pH of the formulation as initially prepared. In some embodiments, the pH of the formulation changes by less than about 0.2 relative to the pH of the formulation as initially prepared. The pH may be measured using, for example, a pH meter. Examples of stable chlorite formulations include those in which less than any of about 0.1%, less than about 0.2%, less than about 0.3%, less than about 0.4%, less than about 0.5%, less than about 0.6%, less than about 0.7%, less than about 0.8%, less than about 0.9%, less than about 1%, less than about 2%, less than about 3%, less than about 4%, less than about 5%, less than about 6%, less than about 7%, less than about 8%, less than about 9%, or less than about 10% of the chlorite degrades into a non-chlorite ion relative to the amount of chlorite present in the formulation as initially prepared. In some embodiments, less than about 2% of the chlorite degrades into a non-chlorite compound relative to the amount of chlorite present in the formulation as initially prepared. In some embodiments, less than about 0.5% of the chlorite degrades into a non-chlorite compound relative to the amount of chlorite present in the formulation as initially prepared. The presence of non-chlorite elements may be measured, for example, using gas chromatography (GC), mass spectrometry, or other methods known by those of skill in the art.

In some embodiments, the chlorite formulations described herein comprise no greater than about 5% by weight of deleterious non-chlorite elements of other commercially available formulations. In some embodiments, the chlorite formulations described herein comprise any of no greater than about 4%, about 3%, about 2%, about 1%, about 0.5%, about 0.3%, about 0.25%, about 0.2%, about 0.1%, about 0.05%, or about 0.02%, by weight of deleterious non-chlorite elements of other commercially available formulations. In some embodiments, the chlorite formulations described herein comprise any of no greater than about 4% by weight of deleterious non-chlorite elements of other commercially available formulations. In some embodiments, the chlorite formulations described herein comprise any of no greater than about 2% by weight of deleterious non-chlorite elements of other commercially available formulations. In some embodiments, the chlorite formulations described herein comprise any of no greater than about 0.5% by weight of deleterious non-chlorite elements of other commercially available formulations. In some embodiments, the chlorite formulations described herein comprise any of no greater than about 0.05% by weight of deleterious non-chlorite elements of other commercially available formulations. In some embodiments, the chlorite formulations described herein are substantially free of the deleterious non-chlorite elements of other commercially available formulations. Non-limiting examples of methods of detection of non-chlorite components include HPLC; SPCS, for example using a Novosep A2 column with 36 mM Sodium Carbonate as a mobile phase, 5µ, 250×4.0 mm, flow rate 0.8 mL/min; DS-Plus Suppressor, for example using a Novosep A2 column with 3.6 mM Sodium Carbonate as a mobile phase, 5µ, 250×4.0 mm, flow rate 0 8 mL/min; an Allsep A-2 Anion column using 2.1 mM NaHCO sub.3/1.6 mM Na.sub.2CO₃ as a mobile phase, 100×4.6 mm, flow rate 2.0 mL/min; an anion HC column using 2.8 mM NaHCO sub 3:2.2 mM Na₂CO₃ in 10% Methanol as a mobile phase, 150×4.6 mm, flow rate 1.4 mL/min; or an Allsep A-2 Anion column using 2.1 mM NaHCO₃/1.6 mM Na₂CO₃ as a mobile phase, 5µ, 100×4.6 mm, flow rate 1.0 mL/min. See, for example, the Al1tech Associates, Inc. Grace Davison line of products and product information for details In some embodiments, formulations described herein comprise 1% or less, 0.5% or less, 0.1% or less, 0.05% or less w/v of chlorate ions.

The chlorite compositions described herein are substantially free of deleterious non-chlorite components. Examples of deleterious non-chlorite components include non-chlorite components that cause an adverse reaction when administered to physiological systems. In some variations, a deleterious non chlorite component is associated with one or more indicia of toxicity in one or more of in vitro or in vivo assays known in the art, or are associated with one or indicia of toxicity when administered to a physiological system, including but not limited to a subject, including but not limited to a human subject. Deleterious non-chlorite components include but are not limited to sulfate, chlorine dioxide, chlorate, and borate In some embodiments, the chlorite formulations described herein are substantially free of the deleterious non-chlorite elements. In some variations, the chlorite formulations described herein are substantially free of sulfate and chlorate ions.

In some embodiments, the chlorite formulations described herein contain less than 1.9% of chloride ions. In some embodiments, the chlorite formulation contains any of 1.9% or less, 1.8% or less; 1.5% or less; 1.0% or less; 0.5% or less; 0.3% or less; 0.1% or less; 0 05% or less; 0.01% or less; 0.001% or less; from 0.001 to 0.1%; from 0.1 to 0.5%; from 0.5 to 1.0%; from 1.0 to 1.5%; or from 1.5 to 1.8% by weight of chloride ions. In some embodiments, the chlorite formulation contains less than about 0.5% by weight of chloride ions. In some embodiments, the chlorite formulation contains less than about 0.24% by weight of chloride ions In some embodiments, the chlorite formulation contains less than about 0.2% by weight of chloride ions. In some embodiments, the chlorite formulation contains less than about 0.1% by weight of chloride ions. In some embodiments, the chlorite formulation is substantially free of chloride ions. In some embodiments, the level of chloride ions is below the level of detection using HPLC.

In some embodiments, the chlorite formulation contains less than about 1.5% of chlorate ions. In some embodiments, the chlorite formulation contains any 1.4% or less, 1.3% or less; 1.0% or less; 0.5% or less; 0.3% or less; 0.1% or less; 0.05% or less; 0.01% or less; 0.001% or less; from 0.001 to 0.1%; from 0.001 to 0.01%; from 0.01 to 0.1%; of chlorate ions. In some embodiments, the chlorite formulation is substantially free of chlorate ions. In some embodiments, the chlorite formulation contains less than about 0.5% by weight of chlorate ions. In some cases, the chlorite formulation contains 0.05% or less by weight of chlorate ions In some variations, the chlorite formulation is substantially free of chlorate ions. In some embodiments, the chlorite formulation contains less than about 0.05% by weight of chlorate ions. In some embodiments, the chlorite formulation contains less than about 0.01% by weight of chlorate ions. In some embodiments, the level of chlorate ions is below the level of detection using HPLC.

In some embodiments, the chlorite formulation contains less than about 0.7% of sulfate ions. In some embodiments, the chlorite formulation contains any of 0.65% or less; 0.6% or less; 0.5% or less; 0.4% or less; 0.3% or less; 0.2% or less: 0.1% or less; 0.08% or less; 0.07% or less; 0.06% or less; 0.05% or less; 0.005% or less; 0.0005% or less, from 0.001 to 0.1%; from 0.01 to 0.1%; from 0.01 to 0.5%; from 0.06 to 0.08%; or from 0.5 to 0.65% of sulfate ions. In some embodiments, the chlorite formulation contains from 0.5 to about 0.65% of sulfate ions In some embodiments, the chlorite formulation is substantially free of sulfate ions. In some embodiments, the chlorite formulation contains less than about 0.5% by weight of sulfate ions. In some embodiments, the chlorite formulation is substantially free of sulfate ions. In some embodiments, the chlorite formulation contains less than about 0.08% by weight of sulfate ions. In some embodiments, the level of sulfate ions is below the level of detection using HPLC.

In some embodiments, the chlorite formulations described herein comprise phosphate ions. In some embodiments, the chlorite formulations described herein comprise sodium ions. In some embodiments, a chlorite formulation comprises chlorite, an aqueous solvent, sodium, and phosphate ions. In some variations, the aqueous solvent consists essentially of water. In some embodiments, a chlorite formulation consists essentially of chlorite, water, sodium, and phosphate, and is substantially free of chlorate. In some embodiments, a chlorite formulation consists essentially of chlorite, water, sodium, and phosphate, and is substantially free of chlorate, and further comprises a pharmaceutically acceptable diluent. In some embodiments, sodium and phosphate are provided in whole or in part as monosodium phosphate or disodium phosphate In some embodiments, the pharmaceutically acceptable diluent is a saline solution.

In some embodiments, the chlorite formulations described herein comprise no greater than 10% by weight of by products or impurities present in commercially available technical grade chlorite. Non-limiting examples of by-products or impurities present in commercially available technical grade chlorite include chlorate, sulfate, chlorine dioxide, chloride, sodium bicarbonate, and sodium carbonate. In some embodiments, the chlorite formulations described herein comprise no greater than about any of 15%, about 12%, about 10%, about 9%, about 8%, about 7%, about 6%, about 5%, about 4%, about 3%, about 2%, about 1%, about 0.5%, about 0.3%, about 0.1%, about 0.05%, from 0.05 to 5%; from 5 to 10%; or from 10 to 15% by weight of one or more degradation products or impurities present in commercially available technical grade chlorite, including but not limited to one or more of chlorate or sulfate In some embodiments, the chlorite formulations described herein comprise no greater than about 0.5% by weight of degradation products or impurities present in commercially available technical grade chlorite, including but not limited to one or more of chlorate or sulfate. In some embodiments, the chlorite formulations described herein comprise no greater than about 5% by weight of degradation products or impurities present in commercially available technical grade chlorite, including but not limited to one or more of chlorate or sulfate. In some embodiments, the chlorite formulations described herein are substantially free of the degradation products or impurities present in commercially available technical grade chlorite, including but not limited to chlorate or sulfate.

Various methods can be used to adjust the pH of formulations and pharmaceutical formulations comprising chlorite. It is intended that the methods described herein can be used to produce the formulations or pharmaceutical formulations described herein for use with the present invention. However, the formulations and pharmaceutical formulations described herein may also be produced by other methods, and the formulations and pharmaceutical formulations described herein are not limited to those produced by the methods described herein.

Some compounds or formulations are sensitive to high local acidity or alkalinity, requiring proper methods to adjust the pH of such compounds or formulations Preferred pH adjusting agent(s) or pH adjusting compound(s) are weak acids or weak bases having a pKa of about 4 to about 9, a pKa of about 5 to about 9, or a pKa of about 5 to about 8, or a pKa of about 6 to about 7.5. Examples include, but are not limited to a phosphate buffer having a pKa of about 4 to about 9 as well known in the field, for example, monobasic phosphates, or monosodium phosphate and/or disodium phosphate and lower alkanoic acids, for example, acetic acid or propionic acid. In some embodiments, the pH of a formulation sensitive to acidity is lowered to from 7 to 11.5 using a pH adjusting compound that does not expose the formulation to acidity, including but not limited to a high local acidity in the area around the pH adjusting compound. In some embodiments, the pH of a formulation sensitive to acidity is lowered to a pH from 8 to 9 using a pH adjusting compound that does not expose the formulation to acidity, including but not limited to a high local acidity in the area around the pH adjusting compound. In some embodiments, the pH of a formulation sensitive to acidity is lowered to a pH from 7.5 to 9.5 using a pH adjusting compound that does not expose the formulation to acidity, including but not limited to a high local acidity in the area around the pH adjusting compound. In some embodiments, the pH of a formulation sensitive to acidity is lowered to a pH from 7 to 9.0 using a pH adjusting compound that does not expose the formulation to acidity, including but not limited to a high local acidity in the area around the pH adjusting compound. In some embodiments, the pH of a formulation sensitive to acidity is lowered to a pH from 7.5 to 8.5 using a pH adjusting compound that does not expose the formulation to acidity, including but not limited to a high local acidity in the area around the pH adjusting compound. In some embodiments, the pH of a formulation sensitive to acidity is lowered to pH from 8.1 to 8.9 using a pH adjusting compound that does not expose the formulation to acidity, including but not limited to a high local acidity in the area around the pH adjusting compound

“High local acidity,” as used herein, refers to the pKa of one or more molecules local to a chlorite molecule, as opposed to the overall acidity of a solution as would be measured, for example, using a pH meter. To determine whether a pH-adjusting agent will subject chlorite to high local acidity, the pKa of the pH adjusting agent can be identified using, for example, the CRC Handbook of Chemistry and Physics (86th Edition, David R. Lide ed., CRC Press, 2005).

Lowering the pH of chlorite formulations has been challenging because many pH adjusting agents expose compounds or formulations to high acidity in the local area of the molecules of the pH-adjusting compound. In the presence of high local acidity, some amount of non-chlorite compounds are generated, e.g., chlorate and/or chlorine dioxide. See, e.g., Ullmann’s Encyclopedia of Industrial Chemistry, Vol. A6, Ed. Wolfgang Gerhartz, 5th Ed. (1986), which is incorporated herein by reference in its entirety. Such degradation products may not be desired in formulations for parenteral or systemic administration to physiological systems, e.g., because they are not inactive in physiological systems. Some such degradation products result in toxicity, including but not limited to the toxicities, including but not limited to non-specific toxicity, described herein.

In some variations, the activity of a therapeutic agent, including but not limited to chlorite, is diminished by exposure to high local acidity. “Diminished activity,” as used herein, refers to an activity of a therapeutic agent that is qualitatively or quantitatively inferior to that of the therapeutic agent prior to the exposure to high local acidity. As one example, a changed activity that is qualitatively or quantitatively inferior to that of the therapeutic agent prior to the exposure to high local acidity would be a lesser efficacy of wound healing, or a lesser efficacy in treating one or more of the diseases or conditions described herein. In some variations, the changed activity is any of at least about 3%, at least about 5%, at least about 10%, at least about 15%, at least about 20%, or at least about 25% lower than the activity of the therapeutic agent prior to the exposure to high local acidity. In some variations, the changed activity is at least about 5% lower than the activity of the therapeutic agent prior to the exposure to high local acidity.

In some embodiments, the pH of a chlorite formulation is adjusted to any one or more of the pH levels described in the formulations section or elsewhere herein. In some embodiments, the pH of a chlorite formulation described from 7 to 11.5. In some embodiments, the method comprises lowering the pH of a formulation comprising chlorite to any of from to 11; from 7 to 10 5, from 7 to 10; from 7 to 9.5; from 7 to 9; from 7 to 8.5; from 7 to 8.0; from 7 to 7.5; from 7.5 to 8; from 7.5 to 8 5; from 7 to 8; from 7.1 to 7.7; from 7.2 to 7.6; from 7.3 to 7.5; from 8 to 9; from 8 to 8.5; from 8.5 to 9; about 7.0, about 7.1; about 7.2, about 7.3; about 7.4; about 7.5; about 7.6; about 7.7; about 7.8; about 7.9; about 8.0; about 8.1; about 8.2; about 8.3; about 8.4; about 8.5; about 8.6; about 8.7, about 8.8; about 8.9; or about 9.0 using a pH adjusting agent that does not expose the chlorite to a high local acidity. In some embodiments, the method comprises lowering the pH of a formulation comprising chlorite to from 8 to 9. In some embodiments, the method comprises lowering the pH of a formulation comprising chlorite to from 7 to 8.5. In some embodiments, the method comprises lowering the pH of a formulation comprising chlorite to from 7 to 8.0. In some embodiments, the method comprises lowering the pH of a formulation comprising chlorite to from 8.1 and about 8.9 In some embodiments, the method comprises lowering the pH of a formulation comprising chlorite to from 7.1 and about 7.9. In some embodiments, the method comprises lowering the pH of a formulation comprising chlorite to about 7.4.

In one non-limiting example, the pH of a mixture comprising chlorite is adjusted using a pH adjusting agent that does not subject the chlorite to a local pH of below 7 when exposed to the mixture comprising chlorite. In some embodiments, the pH adjusting agent is monosodium phosphate, disodium phosphate, or a mixture thereof. In some embodiments, monosodium phosphate and/or disodium phosphate is used as a solid or in solution. In some embodiments, the pH adjusting agent is acetic acid.

In some embodiments, the pH of chlorite is adjusted by adding chlorite or an aqueous mixture comprising chlorite to a solution containing buffer. In some embodiments, the pH of chlorite is adjusted by adding chlorite or an aqueous mixture comprising chlorite to a solution of a phosphate buffer.

In some variations, one or more pH-adjusting agents are used to adjust the pH of a chlorite solution or mixture, and the resulting solution or mixture is analyzed for the presence of degradation products of chlorite, including but not limited to degradation products generated by high local acidity. In some variations, pH-adjusting agents such as acetic acid, monosodium phosphate, and/or disodium phosphate are used to adjust the pH of a chlorite solution or mixture, and the resulting solution or mixture is analyzed for the presence of chlorate or chlorine dioxide

In some embodiments, the resulting solution or mixture is analyzed for degradation products using well known analytical methods such as HPLC, mass spectrometry, etc. In some embodiments, the resulting solution or mixture is analyzed for degradation products using a toxicity assay, including well-known toxicity assays. In some embodiments, the resulting solution or mixture is analyzed for impurities using a non-specific toxicity assay.

In some embodiments, the pH of a chlorite formulation is adjusted after a chlorite purification step. In some embodiments, the pH of a chlorite formulation is adjusted to a pH from 7 to 11.5 without the generation of chlorite degradation products that are a result of high local acidity. In some embodiments, the pH of a chlorite formulation is adjusted to a pH from 8 to 9 without the generation of chlorite degradation products that are a result of high local acidity. In some embodiments, the pH of the chlorite formulation is adjusted to a pH of any of from 7 to 11; from 7 to 10.5; from 7 to 10; from 7 to 9.5; from 7 to 9; from 7 to 8.5; from 7 to 8; from 7 to 7.5; from 7.5 to 8; from 7.5 to 8.5; from 7 to 8; from 8 to 9; from 8 to 8.5, or from 8.5 to 9 without the generation of chlorite degradation products that are a result of high local acidity.

Chlorite-containing compositions, can be formulated for parenteral or enteral administration, generally parenteral administration. Accordingly, formulations of chlorite are suitable for parenteral, topical, or transdermal administration, usually intravenous, intramuscular, or subcutaneous administration, and may be suitable for administration by bolus injection, sustained release (including controlled release), infusion, and the like. Administration by infusion (e.g., by subcutaneous or intravenous infusion) is of interest.

The chlorite composition can be administered alone or in various combinations. Where administered in combination, the chlorite composition can be administered in conjunction with other agents, particularly those suitable for protective, palliative or supportive care of the subject. The phrase “in conjunction with” means that an agent is administered prior to, concurrently, or after other substance or therapy. Examples of agents for administration in conjunction with an agent include, but are not limited to, remdesivir. Other agents for administration in conjunction with chlorite include agents for control of symptoms of a macrophage-associated respiratory disorder, such as ARDS, e.g., as can be associated with COVID-19 symptoms. Further example agents for administration in conjunction with chlorite according to the invention include, but are not limited to, steroids, such as dexamethasone. Chlorite can also be administered in conjunction with non-drug therapy (e.g., physical and/or occupational therapy, massage, and the like).

In one embodiment, the composition does not contain an amount of another anti-proliferative agent, effective to decrease the level of abnormal macrophages in a macrophage-associated respiratory disorder patient, such as an ARDS patient, e.g., as can be associated with a COVID-19 patient (e.g., as compared to prior to therapy). The present invention contemplates that chlorite ions (e.g., as a pharmaceutically acceptable salt in a composition as described herein) are administered to a macrophage-associated respiratory disorder patient, such as an ARDS patient, e.g., as can be associated with a COVID-19 patient so that the chlorite is the active ingredient present in the subject in an amount effective to facilitate treatment of the patient e.g., through reduction in proliferating/inappropriately activated macrophages, and without the need for administration of any other anti-proliferative agent in conjunction with chlorite.

### Administration and Dosing

Chlorite formulations are generally dosed in vivo corresponding to the body weight of the subject Due to the continuous breakdown of the active agent in the blood, the agent is normally administered at regular intervals. Those of skill in the art will readily appreciate that actual dosages and regimen will vary as a function of the agent, formulation, the severity of the symptoms, the susceptibility of the subject to treatment and/or side effects, and the like. Dosages are readily and routinely determinable by those of skill in the art by a variety of means.

Unless the context indicates otherwise, all of the formulations and pharmaceutical formulations described herein may be administered by any of systemic, parenteral (e.g., intramuscular, intraperitoneal, intravenous, ICV, intracisternal injection or infusion, subcutaneous injection, or implant), by inhalation spray, nebulized or aerosolized using aerosol propellants, nasal, vaginal, rectal, sublingual, urethral (e.g., urethral suppository), by infusion, intraarterial, intrathecal, intrabronchial, subcutaneous, intradermal, intravenous, intracervical, intraabdominal, intracranial, intrapulmonary, intrathoracic, intratracheal, nasal routes, oral administration that delivers the therapeutic agent systemically, drug delivery device, or by a dermal patch that delivers the therapeutic agent systemically, transdermally or transbuccally.

In some embodiments, the formulations, pharmaceutical formulations, and methods of administration and treatment described herein are suitable for use in any vertebrate, such as warm- or cold-blooded animal. In some embodiments, the formulations, pharmaceutical formulations, and methods of administration and treatment described herein are suitable for use in a mammal, including in the veterinary context, including domestic pets (such as cats, dogs, rabbits, birds, horses, etc.) and agricultural animals (such as bovine, ovine, fowl, etc.). In some variations, the formulations, pharmaceutical formulations, and methods of administration and treatment described herein are suitable for use in primates, including but not limited to humans.

Example doses of the subject chlorite compositions can vary between about 0.1 mg/kg to about 1.5 mg/kg, such as, about 0.5 mg/kg of body weight and at a concentration of about 40 to about 80 mMol ClO₂⁻ per liter, such as about 60 mMol ClO₂⁻ per liter, respectively. In some cases, doses of the chlorite compositions can vary from 1 to 5 mg/kg of body weight per day, such as about 1 or more mg/kg per day, about 2 or more mg/kg per day, about 3 or more mg/kg per day, about 4 or more mg/kg per day, or about 5 mg/kg per day. In some cases, the doses of the chlorite composition can be from about 1 to 2 mg, from about 2 to 3 mg, from about 3 to 4 mg, or from about 4 to 5 mg per kg of body weight per day.

The regimen of administration (e.g., dose combined with frequency of administration) will generally involve administration in an amount and at a frequency to provide for a desired effect, e.g., administration of an amount effective to provide for improvement in one or more symptoms of a macrophage-associated respiratory disorder patient, such as one or more ARDS symptoms, e.g., as can be associated with one or more COVID-19 symptoms. For example, chlorite can be administered for 2, 3, 4, 5, 6, 7, 8, 9, 10 or more consecutive days, which administration period may be reinitiated after 1, 2, 3 or more weeks following the last dose. In one embodiment, a chlorite treatment regimen comprises 5-7 consecutive days of treatment for a single cycle. In one embodiment, the chlorite treatment regime comprises treatment with 1-5 mg/kg of body weight of the chlorite composition for 5 consecutive days. In one embodiment, the chlorite treatment regime comprises treatment with 1-5 mg/kg of body weight of the chlorite composition for 6 consecutive days. In another embodiment, the chlorite treatment regime comprises treatment with 1-5 mg/kg of body weight of the chlorite composition for 7 consecutive days.

Chlorite according to the disclosure can be administered on a daily basis. In some embodiments, chlorite is administered on a daily basis at a dose of 0.2 mg/kg/day of chlorite to 5.0 mg/kg/day of chlorite. In some embodiments, chlorite is administered on a daily basis at a dose of 0.2 mg/kg/day of chlorite per day, 0.4 mg/kg/day of chlorite per day, 0.5 mg/kg/day of chlorite, 0.6 mg/kg/day of chlorite, 0.7 mg/kg/day of chlorite, 0.8 mg/kg/day of chlorite, 0.9 mg/kg/day of chlorite, 1.0 mg/kg/day of chlorite, 1.1 mg/kg/day of chlorite, 1.2 mg/kg/day of chlorite, 1.3 mg/kg/day of chlorite, 1.4 mg/kg/day of chlorite, 1.5 mg/kg/day of chlorite, 1.6 mg/kg/day of chlorite, 1.7 mg/kg/day of chlorite, 1.8 mg/kg/day of chlorite, 1.9 mg/kg/day of chlorite, 2.0 mg/kg/day of chlorite, 2.1 mg/kg/day of chlorite, 2.2 mg/kg/day of chlorite, 2.3 mg/kg/day of chlorite, 2.4 mg/kg/day of chlorite, 2.5 mg/kg/day of chlorite, 2.6 mg/kg/day of chlorite, 2.7 mg/kg/day of chlorite, 2.8 mg/kg/day of chlorite, 2.9 mg/kg/day of chlorite, 3.0 mg/kg/day of chlorite, 3.1 mg/kg/day of chlorite, 3.2 mg/kg/day of chlorite, 3.3 mg/kg/day of chlorite, 3.4 mg/kg/day of chlorite, 3.5 mg/kg/day of chlorite, 3.6 mg/kg/day of chlorite, 3.7 mg/kg/day of chlorite, 3.8 mg/kg/day of chlorite, 3.9 mg/kg/day of chlorite, 4.0 mg/kg/day of chlorite, 4.1 mg/kg/day of chlorite, 4.2 mg/kg/day of chlorite, 4.3 mg/kg/day of chlorite, 4.4 mg/kg/day of chlorite, 4.5 mg/kg/day of chlorite, 4.6 mg/kg/day of chlorite, 4.7 mg/kg/day of chlorite, 4.8 mg/kg/day of chlorite, 4.9 mg/kg/day of chlorite, or 5.0 mg/kg/day of chlorite.

In some embodiments, the pharmaceutical composition used in the methods of the invention can be further administered in a cycle. An example of a cycle is composed of a period of time wherein the composition is administered daily for ap period of time. In some cases, the cycle of time is 5 to 7 days. In some cases, the cycle includes: a) a first period of time wherein the pharmaceutical composition is administered at a first dose for a first number of times; and b) a second period of time wherein the pharmaceutical composition is administered at a second dose for a second number of times In some embodiments, the first period of time is about one week, the first number of times is about five, the second period of time is about two weeks, and the second number of times is zero. In other embodiments, the first period of time is about one week, the first number of times is about three, the second period of time is about one week, and the second number of times is zero. The first dose can be about 0 4 mg/kg/day of chlorite to about 5.0 mg/kg/day of chlorite. For example, the first dose can be about 2 mg/kg/day of chlorite. The cycle can be performed multiple times, e.g., about 2, 3, 4, 5, 6, 7, 8, 9, 10 or 10 or more times. In some embodiments, the cycle is performed once. In some embodiments, the cycle is performed about 2-4 times.

In some embodiments, the dosing schedule includes periods of administration alternating with periods of non-administration. For example, chlorite might be administered in a 5-7 day cycle, comprising dosing chlorite each day of the cycle. The cycle could be repeated as necessary to achieve the desired result. In another embodiment, chlorite is administered in a two week cycle, e.g., up to 3 times in a week followed by a week without administration. In some embodiments, a total of 2-4 cycles are performed. In one embodiment, the dosing regimen comprises administration of 1-5 mg/kg/day of chlorite for a total of at least a one week cycle.

In certain embodiments, the chlorite composition can be delivered by intravenous infusion. In certain cases, the dose of chlorite (e.g., as described herein) is delivered by intravenous infusion daily for 5 to 7 consecutive days. In certain cases the intravenous infusion is delivered in 5 hours or less, such as 4 hours or less, 3 hours or less, 2 hours or less or even less. In certain cases, the intravenous infusion of the chlorite composition is delivered over 1 to 5 hours, such as 1 to 3, 1 to 2, or 1 to 1.5 hours. In some cases, the intravenous infusion of the chlorite composition is delivered over about 1 hour.

### Methods

### Methods of Macrophage Modulation

As summarized above, aspects of the methods include a method of regulating macrophage activity in a subject having, or at risk of developing, an acute respiratory distress syndrome (ARDS). The method includes contacting a macrophage of a subject having, or at risk of developing, an acute respiratory distress syndrome (ARDS), with a composition comprising an amount of a chlorite composition effective to regulate one or more functional properties of the macrophage.

In certain embodiments, the one or more functional properties of the macrophage includes any of, macrophage activation, a thrombotic process, a complement activation process, microbial translocation, a non-phagocytic phenotype, an inflammatory factor, and a monocytic migration process into a diseased tissue. In some cases, the one or more functional properties of the macrophage includes a soluble plasma factor associated with cellular activation, inflammation and infiltration of bacteria. In some cases, the method is effective to regulate macrophage activation. In some cases, the method is effective to regulate a thrombotic process. In some cases, the method is effective to regulate a complement activation process. In some cases, the method is effective to regulate a microbial translocation. In some cases, the method is effective to regulate a non-phagocytic phenotype. In some cases, the method is effective to regulate an inflammatory factor. In some cases, the method is effective to regulate a monocytic migration process into a diseased tissue. In some cases, the method is effective to regulate a soluble plasma factor associated with cellular activation. In some cases, the method is effective to regulate a soluble plasma factor associated with infiltration of bacteria.

In some embodiments of the methods of regulating macrophage activity in a subject, the functional property is inappropriate macrophage activation, and the contacting results in the regulation of the macrophage activation.

In one embodiment, regulation of inappropriate macrophage activity results in modulation of macrophage proliferation, e.g., alteration of the level of proliferating macrophages or the rate of macrophage proliferation compared to in the absence of agent administration, and/or to effect modulation of inappropriate macrophage activation. An effective amount of chlorite composition (e.g., as described herein) is determined by, for example, comparing the level (or number) of promacs, before and during treatment, with a downward trend in the number of promacs generally being consistent with a positive effect. In one embodiment, the chlorite composition is administered so as to effect a change in the level of proliferating macrophages or the rate of macrophage proliferation of at least 25%, such as at least 50%, at least 75%, or at least 90%. The degree of modulation may be assessed by measurement of macrophage proliferation as described in the art, and generally entails detecting a proliferation marker(s) in a macrophage population or uptake of certain substances such as BrdU or 3H-thymidine (which would provide a quantitative measure of proliferation) (see, e.g., U.S. Publication No. 20030175832). Such a decrease may have desirable concomitant effects, such as to palliate, ameliorate, stabilize, reverse, slow and/or delay progression of disease, delay or even prevent onset of disease.

In another embodiment, contacting a macrophage of a subject with a composition comprising an amount of a chlorite composition is effective to decrease the level (e.g., number) of pathologic macrophages, e.g., to effect a decrease in the level of CD14+ monocytes, such as activated CD14+ monocytes, in a patient with a macrophage-associated respiratory disorder (e.g., a patient with ARDS, e.g., as can be associated with COVID-19). In certain cases, the method is in vivo. In this embodiment, chlorite is administered in an amount sufficient to decrease the level of (e.g., number of) CD14+ monocytes, such as activated CD14+ monocytes and/or CD14+ monocytes with elevated HLA-DR expression and/or the number of CD14+/CD16+ cells and/or the percentage of CD16+ cells in a population of CD14+ cells in the individual (i.e., an effective amount). An effective amount of chlorite is determined by, for example, comparing the level of number of CD14+ monocytes, such as activated CD14+ monocytes, before and during treatment, with a downward trend of number of CD14+ monocytes generally being consistent with a positive effect. An “amount sufficient to decrease the number of CD14+ monocytes” is able to decrease the number of CD 14+ monocytes by at least about 25%, such as at least about 50%, at least about 75%, or at least about 90%. Methods for assessing levels of CD14+ monocytes, activated CD14+ monocytes, CD14+ monocytes with elevated HLA-DR expression, CD14+/CD16+ cells and the percentage of CD16+ cells in a population of CCD14+ are known in the art (see, e.g., U.S. Publication No. 20030175832). Such a decrease may have desirable concomitant effects, such as to palliate, ameliorate, stabilize, reverse, slow and/or delay progression of disease, delay or even prevent onset of disease.

Levels of pathologic macrophages (proliferating/inappropriate activated macrophages (promacs)), macrophage proliferation rate, CD14+ cells, HLA-DR expression, and the like as set out above can be compared to a level from the same individual measured at a different time and/or under different conditions (such as before treatment, different dose, etc.), and/or to a mean or median level determined for a non-diseased standard (e.g., non- macrophage-associated respiratory disorder patient), for example from an unaffected individual (e.g., non- macrophage-associated respiratory disorder individual or individuals; a non-ARDS individual or non-ARDS individuals; or non-COVID-19 individual or non-COVID-19 individuals).

For example, an HLA-DR expression level may be compared to an HLA-DR level from the same individual measured at a different time and/or under different conditions (such as before treatment, different dose, etc.). In some embodiments, an HLA-DR expression level is compared to a mean or median level of HLA-DR expression determined on a population of CD14+ cells from a non-diseased (e.g., non-ARDS, or non-COVID-19) standard, for example from a non-ARDS individual or non-ARDS individuals, or non-COVID-19 individual or non-COVID-19 individuals). A finding of HLA-DR expression level of greater than about 1.4 fold that of the non-diseased standard is indicative of an elevated level of HLA-DR expression in the individual. Generally, a finding of HLA-DR expression level of greater than about 1.5 fold, greater than about 1.6 fold, greater than about 1.7 fold, greater than about 1.8 fold, greater than about 1.9 fold, greater than about 2.0 fold, greater than about 5.0 fold, or greater than about 10 fold that of a non-diseased standard is indicative of an elevated level of HLA-DR expression in the individual. Thus, decreasing HLA-DR expression in a macrophage-associated respiratory disorder subject (e.g., an ARDS subject, or a COVID-19 subject) so as to more closely approximate an HLA-DR expression level in a non-diseased subject (e.g., non-ARDS, or non-COVID-19 subject) is of interest in the present disclosure.

In another example, the number of CD14+/CD16+ cells or the percentage of CD16+ cells in a population of CD14+ cells in a sample from a macrophage-associated respiratory disorder subject (e.g., an ARDS subject, or a COVID-19 subject) is compared to a mean or median level of CD14+/CD16+ cells in a biological sample from a non-disease (e.g., non-ARDS, or non-COVID-19) standard, for example from a non-ARDS individual or non-ARDS individuals; or non-COVID-19 individual or non-COVID-19 individuals. A finding of a percentage of CD16+ cells in a population of CD14+ cells and/or the number of CD14+/CD16+ cells in a sample of greater than about 1.5 fold, greater than about 1.6 fold, greater than about 1.7 fold, greater than about 1.8 fold, greater than about 1.9 fold, greater than about 2.0 fold, greater than about 3.0 fold, greater than about 4.0 fold, greater than about 5.0 fold, or greater than about 10 fold that of a non-diseased (non-ARDS, or non-COVID-19) standard is indicative of an increased number of CD14+/CD16+ cells in the individual. Thus, in one embodiment, therapy according to the invention is provided so as to decrease the number of CD14+/CD16+ cells or the percentage of CD16+ cells in a population of CD14+ cells so as to more closely approximate such in an appropriate non-diseased subject.

In general, therapy is monitored by following blood macrophage activation, usually by following CD14/DR levels and the percentage of CD14/16 positive cells as described above.

In some embodiments of the methods of regulating macrophage activity in a subject, the functional property is a soluble plasma factor associated with one or more of cellular activation, inflammation, and infiltration of bacteria.

In some embodiments of the methods of regulating macrophage activity in a subject, the functional property is an inflammatory factor, and the contacting decreases the level of the inflammatory factor activity as measured in the blood.

In some embodiments of the methods of regulating macrophage activity in a subject, the functional property is a factor associated with cellular activation, and the contacting decreases the level of the factor associated with cellular activation activity as measured in the blood.

In some embodiments of the methods of regulating macrophage activity in a subject, the functional property is a factor associated with infiltration of bacteria, and the contacting decreases the level of the factor associated with infiltration of bacteria activity as measured in the blood.

In certain cases, a macrophage of a subject having a macrophage-associated respiratory disorder, such as ARDS, e.g., as associated with COVID-19, is characterized by producing elevated levels of one or more soluble plasma factors associated with cellular activation, inflammation and infiltration of bacteria. The term “elevated levels” refers to a level of one or more soluble plasma factors in blood that is 20% or more than the native or basal level of the soluble plasma factor in a control blood, such as 30% or more, 40% or more, 40% or more, 40% or more, 40% or more, 40% or more, 40% or more, 2-fold greater or more, 5-fold greater or more, 10-fold greater or more, 30-fold greater or more, 100-fold greater or more or 1000-fold greater or more, as compared to the native or basal level of the soluble plasma factor in control blood. In certain cases, the control blood value is from a plurality of subjects. In some cases, the control blood values are those present in normal blood.

In certain cases of the methods of regulating macrophage activity, contacting the macrophage of a subject having a macrophage-associated respiratory disorder (such as ARDS, e.g., as can be associated with COVID-19) with a subject chlorite composition, decreases the level of the soluble plasma factor activity as measured in the blood. The chlorite composition (e.g., as described herein) may decrease the level of one or more soluble plasma factors in the range of 10% to 100%, e.g., by 10% or more, 20% or more, 30% or more, 40% or more, 50% or more, 60% or more, 70% or more, 80% or more, or 90% or more relative to the level of the soluble plasma factor before contacting with the chlorite composition. In certain cases, contacting the macrophage of a subject having a macrophage-associated respiratory disorder (such as ARDS, e.g., as can be associated with COVID-19) with a subject chlorite composition, decreases the level of the soluble plasma factor activity as measured in the blood, to a native or basal level (e.g., the level in control blood).

In certain cases of the methods of regulating macrophage activity, the soluble plasma factor is selected from one or more of interferon gamma (IFN-y), interleukin-1 receptor antagonist (IL-1Ra), interleukin-2 receptor antagonist (IL-2Ra), interleukin-6 (IL-6), interleukin-10 (IL-10), interleukin-18 (IL-18), hepatocyte growth factor (HGF), monocyte chemotactic protein 3 (MCP-3), monokine induced by gamma interferon (MIG, also known as and referred to herein as CXCL9), macrophage colony-stimulating factor (M-CSF), granulocyte colony-stimulating factor (G-CSF), macrophage inflammatory protein-1a (MIP-1a), interferon gamma-induced protein 10 (IP-10, also known as and referred to herein as CXCL10), LPS-binding protein (LBP), bactericidal permeability increasing protein (BPI), sCD14, sCD163, Osteopontin (OPN), Calprotectin (S100A8/9), monocyte chemoattractant protein-1 (MCP-1), or a combination thereof.

In certain cases of the methods of regulating macrophage activity, the soluble plasma factor is selected from one or more of C-X-C motif chemokine ligand 9 (CXCL9), C-X-C motif chemokine ligand 10 (CXCL10), interleukin-1 receptor antagonist (IL1Ra), hepatocyte growth factor (HGF), sCD163, osteopontin (OPN), or a combination thereof.

In certain cases of the methods of regulating macrophage activity, the one or more soluble plasma factors includes C-X-C motif chemokine ligand 9 (CXCL9), and contacting the macrophage with the subject chlorite composition decreases the level of CXCL9 by 10% or more, 20% or more, 30% or more, 40% or more, 50% or more, 60% or more, 70% or more, 80% or more, or 90% or more, relative to the level before contacting with a subject chlorite composition.

In certain cases of the methods of regulating macrophage activity, the one or more soluble plasma factors includes C-X-C motif chemokine ligand 10 (CXCL10), and contacting the macrophage with the subject chlorite composition decreases the level of CXCL10 by 10% or more, 20% or more, 30% or more, 40% or more, 50% or more, 60% or more, 70% or more, 80% or more, or 90% or more, relative to the level before contacting with a subject chlorite composition.

In certain cases of the methods of regulating macrophage activity, the one or more soluble plasma factors includes interleukin-1 receptor antagonist (IL1Ra), and contacting the macrophage with the subject chlorite composition decreases the level of ILIRa by 10% or more, 20% or more, 30% or more, 40% or more, 50% or more, 60% or more, 70% or more, 80% or more, or 90% or more., relative to the level before contacting with a subject chlorite composition.

In certain cases of the methods of regulating macrophage activity, the one or more soluble plasma factors includes hepatocyte growth factor (HGF), and contacting the macrophage with the subject chlorite composition decreases the level of HGF by 10% or more, 20% or more, 30% or more, 40% or more, 50% or more, 60% or more, 70% or more, 80% or more, or 90% or more., relative to the level before contacting with a subject chlorite composition.

In certain cases of the methods of regulating macrophage activity, the one or more soluble plasma factors includes sCD163, and contacting the macrophage with the subject chlorite composition decreases the level of sCD163 by 10% or more, 20% or more, 30% or more, 40% or more, 50% or more, 60% or more, 70% or more, 80% or more, or 90% or more., relative to the level before contacting with a subject chlorite composition.

In certain cases of the methods of regulating macrophage activity, the one or more soluble plasma factors includes osteopontin (OPN), and contacting the macrophage with the subject chlorite composition decreases the level of OPN by 10% or more, 20% or more, 30% or more, 40% or more, 50% or more, 60% or more, 70% or more, 80% or more, or 90% or more, relative to the level before contacting with a subject chlorite composition.

In certain embodiments, the subject method is an in vitro method that includes contacting a sample comprising a macrophage from a subject having a macrophage-associated respiratory disorder (such as ARDS, e.g., as can be associated with COVID-19) with a chlorite composition. In certain cases, the sample is suspected of having elevated levels of one or more (or all) of the soluble plasma factors C-X-C motif chemokine ligand 9 (CXCL9), C-X-C motif chemokine ligand 10 (CXCL10), interleukin-1 receptor antagonist (IL1Ra), hepatocyte growth factor (HGF), sCD163, osteopontin (OPN), or any combination thereof and the subject methods further includes evaluating whether the chlorite composition decreases one or more of these soluble plasma factors.

In certain cases, a macrophage of a subject having a macrophage-associated respiratory disorder, such as ARDS, e.g., as associated with COVID-19, is characterized by producing elevated levels of one or more inflammatory factors. The term “elevated levels” refers to a level of one or more inflammatory factors in blood that is 20% or more than the native or basal level of the inflammatory factor in a control blood, such as 30% or more, 40% or more, 40% or more, 40% or more, 40% or more, 40% or more, 40% or more, 2-fold greater or more, 5-fold greater or more, 10-fold greater or more, 30-fold greater or more, 100-fold greater or more or 1000-fold greater or more, as compared to the native or basal level of the inflammatory factor in control blood. In certain cases, the control blood value is from a plurality of subjects. In some cases, the control blood values are those present in normal blood.

In certain cases of the methods of regulating macrophage activity, contacting the macrophage of a subject having a macrophage-associated respiratory disorder (such as ARDS, e.g., as can be associated with COVID-19) with a subject chlorite composition, decreases the level of the inflammatory factor activity as measured in the blood. The chlorite composition (e.g., as described herein) may decrease the level of one or more inflammatory factors in the range of 10% to 100%, e.g., by 10% or more, 20% or more, 30% or more, 40% or more, 50% or more, 60% or more, 70% or more, 80% or more, or 90% or more relative to the level of the inflammatory factor before contacting with the chlorite composition. In certain cases, contacting the macrophage of a subject having a macrophage-associated respiratory disorder (such as ARDS, e.g., as can be associated with COVID-19) with a subject chlorite composition, decreases the level of the inflammatory factor activity as measured in the blood, to a native or basal level (e.g., the level in control blood).

In certain cases of the methods of regulating macrophage activity, the inflammatory factor is selected from one or more of interleukin-18 (IL-18), C-X-C motif chemokine ligand 9 (CXCL9), C-X-C motif chemokine ligand 10 (CXCL10), interleukin-1 receptor antagonist (IL1Ra), osteopontin (OPN), or a combination thereof.

In certain cases of the methods of regulating macrophage activity, the one or more inflammatory factors includes interleukin-18 (IL-18), and contacting the macrophage with the subject chlorite composition decreases the level of IL-18 by 10% or more, 20% or more, 30% or more, 40% or more, 50% or more, 60% or more, 70% or more, 80% or more, or 90% or more, relative to the level before contacting with a subject chlorite composition.

In certain cases of the methods of regulating macrophage activity, the one or more inflammatory factors includes C-X-C motif chemokine ligand 9 (CXCL9), and contacting the macrophage with the subject chlorite composition decreases the level of CXCL9 by 10% or more, 20% or more, 30% or more, 40% or more, 50% or more, 60% or more, 70% or more, 80% or more, or 90% or more, relative to the level before contacting with a subject chlorite composition.

In certain cases of the methods of regulating macrophage activity, the one or more inflammatory factors includes C-X-C motif chemokine ligand 10 (CXCL10), and contacting the macrophage with the subject chlorite composition decreases the level of CXCL10 by 10% or more, 20% or more, 30% or more, 40% or more, 50% or more, 60% or more, 70% or more, 80% or more, or 90% or more, relative to the level before contacting with a subject chlorite composition.

In certain cases of the methods of regulating macrophage activity, the one or more inflammatory factors includes interleukin-1 receptor antagonist (IL1Ra), and contacting the macrophage with the subject chlorite composition decreases the level of ILIRa by 10% or more, 20% or more, 30% or more, 40% or more, 50% or more, 60% or more, 70% or more, 80% or more, or 90% or more., relative to the level before contacting with a subject chlorite composition.

In certain cases of the methods of regulating macrophage activity, the one or more inflammatory factors includes osteopontin (OPN), and contacting the macrophage with the subject chlorite composition decreases the level of OPN by 10% or more, 20% or more, 30% or more, 40% or more, 50% or more, 60% or more, 70% or more, 80% or more, or 90% or more., relative to the level before contacting with a subject chlorite composition.

In certain embodiments, the subject method is an in vitro method that includes contacting a sample comprising a macrophage from a subject having a macrophage-associated respiratory disorder (such as ARDS, e.g., as can be associated with COVID-19) with a chlorite composition. In certain cases, the sample is suspected of having elevated levels of one or more (or all) of the inflammatory factors interleukin-18 (IL-18), C-X-C motif chemokine ligand 9 (CXCL9), C-X-C motif chemokine ligand 10 (CXCL10), interleukin-1 receptor antagonist (IL1Ra), osteopontin (OPN), or any combination thereof and the subject methods further includes evaluating whether the chlorite composition decreases one or more of these inflammatory factors.

In some cases, the method is effective to regulate nuclear factor- κB (NF-κB) function, a major transcription factor that regulates genes responsible for both the innate and adaptive immune response and serves as an important mediator of inflammatory responses.

In certain cases, the methods is effective to inhibit nuclear factor- κB (NF-κB) activity, and contacting the macrophage with the subject chlorite composition inhibits NF-κB activity by 10% or more, 20% or more, 30% or more, 40% or more, 50% or more, 60% or more, 70% or more, 80% or more, or 90% or more., relative to the level of activity before contacting with a subject chlorite composition.

In certain embodiments of the methods of regulating macrophage activity, the functional property is microbial translocation or a monocytic migration process into a diseased tissue. Macrophage produced osteopontin, is the most potent of all monocyte migration inducing factors. The tissues that result from this process have infected macrophages, and recent blood derived monocyte migrants expressing CD 163.

In certain cases of the methods of regulating macrophage activity, contacting the macrophage of a subject having a macrophage-associated respiratory disorder (such as ARDS, e.g., as can be associated with COVID-19) with a subject chlorite composition, inhibits microbial translocation or a monocytic migration process into a diseased tissue (e.g., by decreasing the plasma or serum level of soluble CD163 (sCD163)). The chlorite composition (e.g., as described herein) may decrease the level of sCD163, in the range of 10% to 100%, e.g., by 10% or more, 20% or more, 30% or more, 40% or more, 50% or more, 60% or more, 70% or more, 80% or more, or 90% or more relative to the level of CD163 before contacting with the chlorite composition. In certain cases, contacting the macrophage of a subject having a macrophage-associated respiratory disorder (such as ARDS, e.g., as can be associated with COVID-19) with a subject chlorite composition, decreases the level of sCD163, to a native or basal level (e.g., the level in a control blood).

In certain cases, the method inhibits microbial translocation or a monocytic migration process into a diseased tissue (e.g., by decreasing the plasma or serum level of soluble bactericidal/permeability increasing protein (BPI)). The chlorite composition (e.g., as described herein) may decrease the level of BPI, in the range of 10% to 100%, e.g., by 10% or more, 20% or more, 30% or more, 40% or more, 50% or more, 60% or more, 70% or more, 80% or more, or 90% or more relative to the level of BPI before contacting with the chlorite composition. In certain cases, contacting the macrophage of a subject having a macrophage-associated respiratory disorder (such as ARDS, e.g., as can be associated with COVID-19) with a subject chlorite composition, decreases the level of BPI, to a native or basal level (e.g., the level in a control blood).

In certain cases, the method inhibits microbial translocation or a monocytic migration process into a diseased tissue (e.g., by decreasing the plasma or serum level of soluble LPS binding protein (LBP)). The chlorite composition (e.g., as described herein) may decrease the level of LBP, in the range of 10% to 100%, e.g., by 10% or more, 20% or more, 30% or more, 40% or more, 50% or more, 60% or more, 70% or more, 80% or more, or 90% or more relative to the level of LBP before contacting with the chlorite composition. In certain cases, contacting the macrophage of a subject having a macrophage-associated respiratory disorder (such as ARDS, e.g., as can be associated with COVID-19) with a subject chlorite composition, decreases the level of LBP, to a native or basal level (e.g., the level in a control blood).

In certain cases, the method inhibits microbial translocation or a monocytic migration process into a diseased tissue (e.g., by decreasing the plasma or serum level of soluble Calprotectic (S100A8/9)). The chlorite composition (e.g., as described herein) may decrease the level of one or more of S100A8 and S100A9, in the range of 10% to 100%, e.g., by 10% or more, 20% or more, 30% or more, 40% or more, 50% or more, 60% or more, 70% or more, 80% or more, or 90% or more relative to the level of S100A8 and S100A9 respectively before contacting with the chlorite composition. In certain cases, contacting the macrophage of a subject having a macrophage-associated respiratory disorder (such as ARDS, e.g., as can be associated with COVID-19) with a subject chlorite composition, decreases the level of S100A8/9, to a native or basal level (e.g., the level in a control blood).

In certain cases, the method inhibits microbial translocation or a monocytic migration process into a diseased tissue (e.g., by decreasing the plasma or serum level of sCD14). The chlorite composition (e.g., as described herein) may decrease the level of sCD14, in the range of 10% to 100%, e.g., by 10% or more, 20% or more, 30% or more, 40% or more, 50% or more, 60% or more, 70% or more, 80% or more, or 90% or more relative to the level of sCD14 before contacting with the chlorite composition. In certain cases, contacting the macrophage of a subject having a macrophage-associated respiratory disorder (such as ARDS, e.g., as can be associated with COVID-19) with a subject chlorite composition, decreases the level of sCD14, to a native or basal level (e.g., the level in a control blood).

Kits with unit doses of the subject compounds, usually in injectable doses, are provided. In such kits, in addition to the containers containing the unit doses will be an informational package insert describing the use and attendant benefits of chlorite in treating a macrophage-associated respiratory disorder subject, such as ARDS, e.g., as can be associated with COVID-19. Such compounds and unit doses are those described herein above.

### Methods of Treating ARDS

In some embodiments, the subject method is an in vivo method that includes administering to a subject an effective amount of a chlorite composition (e.g., as described herein) that specifically modulates the activity of a macrophage of a subject having a macrophage-associated respiratory disorder (such as ARDS, e.g., as can be associated with COVID-19). An “effective amount” is an amount of a compound that, when administered to an individual in one or more doses, in monotherapy or in combination therapy, is effective to modulate macrophage activity by at least about 20%, such as at least about 30%, at least about 40%, at least about 50%, at least about 60%, at least about 70%, at least about 80%, or at least about 90%, compared to the macrophage activity in the individual in the absence of treatment with the chlorite composition, or alternatively, compared to the macrophage activity in the individual before or after treatment with the chlorite composition. In certain cases, the modulating of macrophage activity, is a decrease in macrophage activity.

In certain cases, the methods include administering to a subject an effective amount of a chlorite composition (e.g., as described herein) that specifically decreases the level of one or more soluble plasma factor activities (e.g., as described herein) as measured in the blood of a subject having a macrophage-associated respiratory disorder (such as ARDS, e.g., as can be associated with COVID-19). In this case, an “effective amount” is an amount of a compound that, when administered to an individual in one or more doses, in monotherapy or in combination therapy, is effective to decrease one or more soluble plasma factors by at least about 20%, such as at least about 30%, at least about 40%, at least about 50%, at least about 60%, at least about 70%, at least about 80%, or at least about 90%, compared to the soluble plasma factor level in the individual in the absence of treatment with the chlorite composition, or alternatively, compared to the soluble plasma factor in the individual before or after treatment with the chlorite composition.

In certain cases, “effective amount” is an amount of a compound that, when administered to an individual in one or more doses, in monotherapy or in combination therapy, is effective to return one or more soluble plasma factors to a control level (e.g., a level of the soluble plasma factor of a non-ARDS subject) and/or to reduce the level of the one or more soluble plasma factors relative to a pre-treatment level (a level prior to administration of chlorite therapy). In certain cases of the in vivo methods, the soluble plasma factor is selected from one or more of C-X-C motif chemokine ligand 9 (CXCL9), C-X-C motif chemokine ligand 10 (CXCL10), interleukin-1 receptor antagonist (IL1Ra), hepatocyte growth factor (HGF), sCD163, osteopontin (OPN), or any combination thereof, including assessment of all of these soluble plasma factors. For example, it may be of interest to assess one or more, or all of, levels of C-X-C motif chemokine ligand 9 (CXCL9), C-X-C motif chemokine ligand 10 (CXCL10), interleukin-1 receptor antagonist (IL1Ra), and hepatocyte growth factor (HGF). In may also be of interest to assess levels of sCD163, and osteopontin (OPN).

In certain cases of the methods of treatment of a subject having a macrophage-associated respiratory disorder (such as ARDS, e.g., as can be associated with COVID-19) with a subject chlorite composition, decreases the level of the soluble plasma factor activity as measured in the blood. The chlorite composition (e.g., as described herein) may decrease the level of one or more soluble plasma factors (e.g., as described herein) in the range of 10% to 100%, e.g., by 10% or more, 20% or more, 30% or more, 40% or more, 50% or more, 60% or more, 70% or more, 80% or more, or 90% or more relative to the level of the soluble plasma factor before contacting with the chlorite composition. In certain cases, the methods of treatment of a subject having a macrophage-associated respiratory disorder (such as ARDS, e.g., as can be associated with COVID-19) with a subject chlorite composition, decreases the level of the soluble plasma factor activity as measured in the blood, to a native or basal level.

In certain cases of methods of treatment of a subject, the soluble plasma factor is selected from one or more of C-X-C motif chemokine ligand 9 (CXCL9), C-X-C motif chemokine ligand 10 (CXCL10), interleukin-1 receptor antagonist (IL1Ra), hepatocyte growth factor (HGF), sCD163, osteopontin (OPN), or a combination thereof. Soluble plasma factors can be assessed in any suitable biological sample, e.g., blood or fraction thereof. Assays that allow quantitation of factor levels include but are not limited to those that involve quantitative detection of plasma or serum levels of “factors”. These assays include ELISA or multiplex immunoassays.

In certain cases of the methods of regulating macrophage activity, the one or more soluble plasma factors includes C-X-C motif chemokine ligand 9 (CXCL9), and contacting the macrophage with the subject chlorite composition decreases the level of CXCL9 as measured in the blood by 10% or more, 20% or more, 30% or more, 40% or more, 50% or more, 60% or more, 70% or more, 80% or more, or 90% or more, relative to the level measured in blood before contacting with a subject chlorite composition.

In certain cases of the methods of regulating macrophage activity, the one or more soluble plasma factors includes C-X-C motif chemokine ligand 10 (CXCL10), and contacting the macrophage with the subject chlorite composition decreases the level of CXCL10 as measured in blood by 10% or more, 20% or more, 30% or more, 40% or more, 50% or more, 60% or more, 70% or more, 80% or more, or 90% or more, relative to the level as measured in blood before contacting with a subject chlorite composition.

In certain cases of the methods of regulating macrophage activity, the one or more soluble plasma factors includes interleukin-1 receptor antagonist (IL1Ra), and contacting the macrophage with the subject chlorite composition decreases the level of ILIRa as measured in blood by 10% or more, 20% or more, 30% or more, 40% or more, 50% or more, 60% or more, 70% or more, 80% or more, or 90% or more., relative to the level measured in blood before contacting with a subject chlorite composition.

In certain cases of the methods of regulating macrophage activity, the one or more soluble plasma factors includes hepatocyte growth factor (HGF), and contacting the macrophage with the subject chlorite composition decreases the level of HGF as measured in blood by 10% or more, 20% or more, 30% or more, 40% or more, 50% or more, 60% or more, 70% or more, 80% or more, or 90% or more., relative to the level measured in blood before contacting with a subject chlorite composition.

In certain cases of the methods of regulating macrophage activity, the one or more soluble plasma factors includes sCD163, and contacting the macrophage with the subject chlorite composition decreases the level of sCD163 as measured in blood by 10% or more, 20% or more, 30% or more, 40% or more, 50% or more, 60% or more, 70% or more, 80% or more, or 90% or more., relative to the level measured in blood before contacting with a subject chlorite composition.

In certain cases of the methods of regulating macrophage activity, the one or more soluble plasma factors includes osteopontin (OPN), and contacting the macrophage with the subject chlorite composition decreases the level of OPN as measured in blood by 10% or more, 20% or more, 30% or more, 40% or more, 50% or more, 60% or more, 70% or more, 80% or more, or 90% or more, relative to the level measured in blood before contacting with a subject chlorite composition.

In certain cases, the methods include administering to a subject an effective amount of a chlorite composition (e.g., as described herein) that specifically decreases the level of one or more inflammatory factor activities as measured in the blood of a subject having a macrophage-associated respiratory disorder (such as ARDS, e.g., as can be associated with COVID-19). In this case, an “effective amount” is an amount of a compound that, when administered to an individual in one or more doses, in monotherapy or in combination therapy, is effective to decrease one or more inflammatory factor by at least about 20%, such as at least about 30%, at least about 40%, at least about 50%, at least about 60%, at least about 70%, at least about 80%, or at least about 90%, compared to the inflammatory factor level in the individual in the absence of treatment with the chlorite composition, or alternatively, compared to the inflammatory factor in the individual before or after treatment with the chlorite composition.

In certain cases, “effective amount” is an amount of a compound that, when administered to an individual in one or more doses, in monotherapy or in combination therapy, is effective to return one or more inflammatory factors to a control level (e.g., a level of the inflammatory factor of a non-ARDS subject) and/or to reduce the level of the one or more inflammatory factors relative to a pre-treatment level (a level prior to administration of chlorite therapy). In certain cases of the in vivo methods, the inflammatory factor is selected from one or more of interleukin-18 (IL-18), C-X-C motif chemokine ligand 9 (CXCL9), C-X-C motif chemokine ligand 10 (CXCL10), and interleukin-1 receptor antagonist (IL1Ra), osteopontin (OPN), or any combination thereof, including assessment of all of these inflammatory factors. For example, it may be of interest to assess one or more, or all of, levels of C-X-C motif chemokine ligand 9 (CXCL9), C-X-C motif chemokine ligand 10 (CXCL10), and interleukin-1 receptor antagonist (IL1Ra). In may also be of interest to assess levels of osteopontin (OPN).

In certain cases of the methods of treatment of a subject having a macrophage-associated respiratory disorder (such as ARDS, e.g., as can be associated with COVID-19) with a subject chlorite composition, decreases the level of the inflammatory factor activity as measured in the blood. The chlorite composition (e.g., as described herein) may decrease the level of one or more inflammatory factors in the range of 10% to 100%, e.g., by 10% or more, 20% or more, 30% or more, 40% or more, 50% or more, 60% or more, 70% or more, 80% or more, or 90% or more relative to the level of the inflammatory factor before contacting with the chlorite composition. In certain cases, the methods of treatment of a subject having a macrophage-associated respiratory disorder (such as ARDS, e.g., as can be associated with COVID-19) with a subject chlorite composition, decreases the level of the inflammatory factor activity as measured in the blood, to a native or basal level.

In certain cases of methods of treatment of a subject, the inflammatory factor is selected from one or more of interleukin-18 (IL-18), C-X-C motif chemokine ligand 9 (CXCL9), C-X-C motif chemokine ligand 10 (CXCL10), interleukin-1 receptor antagonist (IL1Ra), osteopontin (OPN), or a combination thereof. Inflammatory markers can be assessed in any suitable biological sample, e.g., blood or fraction thereof. Assays that allow quantitation of factor levels include but are not limited to those that involve quantitative detection of plasma or serum levels of “factors”. These assays include ELISA or multiplex immunoassays

In certain cases of methods of treatment of a subject,, the one or more inflammatory factors includes interleukin-18 (IL-18), and contacting the macrophage with the subject chlorite composition decreases the level of IL-18 as measured in the blood by 10% or more, 20% or more, 30% or more, 40% or more, 50% or more, 60% or more, 70% or more, 80% or more, or 90% or more, relative to the level measured in blood before contacting with a subject chlorite composition.

In certain cases of the methods of regulating macrophage activity, the one or more inflammatory factors includes C-X-C motif chemokine ligand 9 (CXCL9), and contacting the macrophage with the subject chlorite composition decreases the level of CXCL9 as measured in the blood by 10% or more, 20% or more, 30% or more, 40% or more, 50% or more, 60% or more, 70% or more, 80% or more, or 90% or more, relative to the level measured in blood before contacting with a subject chlorite composition.

In certain cases of the methods of regulating macrophage activity, the one or more inflammatory factors includes C-X-C motif chemokine ligand 10 (CXCL10), and contacting the macrophage with the subject chlorite composition decreases the level of CXCL10 as measured in blood by 10% or more, 20% or more, 30% or more, 40% or more, 50% or more, 60% or more, 70% or more, 80% or more, or 90% or more, relative to the level as measured in blood before contacting with a subject chlorite composition.

In certain cases of the methods of regulating macrophage activity, the one or more inflammatory factors includes interleukin-1 receptor antagonist (IL1Ra), and contacting the macrophage with the subject chlorite composition decreases the level of ILIRa as measured in blood by 10% or more, 20% or more, 30% or more, 40% or more, 50% or more, 60% or more, 70% or more, 80% or more, or 90% or more., relative to the level measured in blood before contacting with a subject chlorite composition.

In certain cases of the methods of regulating macrophage activity, the one or more inflammatory factors includes osteopontin (OPN), and contacting the macrophage with the subject chlorite composition decreases the level of OPN as measured in blood by 10% or more, 20% or more, 30% or more, 40% or more, 50% or more, 60% or more, 70% or more, 80% or more, or 90% or more, relative to the level measured in blood before contacting with a subject chlorite composition.

In certain embodiments, the subject method is an in vitro method that includes contacting a blood sample comprising a macrophage from a subject having a macrophage-associated respiratory disorder (such as ARDS, e.g., as can be associated with COVID-19) with a chlorite composition. In certain cases, the blood sample is suspected of having elevated levels of one or more (or all) of the inflammatory factors interleukin-18 (IL-18), C-X-C motif chemokine ligand 9 (CXCL9), C-X-C motif chemokine ligand 10 (CXCL10), interleukin-1 receptor antagonist (IL1Ra), osteopontin (OPN), or any combination thereof and the subject methods further includes evaluating whether the chlorite composition decreases one or more of these inflammatory factors. In certain cases, the blood sample is suspected of having elevated levels of one or more (or all) of the soluble plasma factors C-X-C motif chemokine ligand 9 (CXCL9), C-X-C motif chemokine ligand 10 (CXCL10), interleukin-1 receptor antagonist (IL1Ra), hepatocyte growth factor (HGF), sCD163, osteopontin (OSP), or any combination thereof and the subject methods further includes evaluation whether the chlorite composition decreases one or more of these soluble plasma factors.

In certain cases, the methods include administering to a subject an effective amount of a chlorite composition (e.g., as described herein) that specifically regulate nuclear factor- κB (NF-κB) function, a major transcription factor that regulates genes responsible for both the innate and adaptive immune response and serves as an important mediator of inflammatory responses.

In certain cases, the methods include administering to a subject an effective amount of a chlorite composition (e.g., as described herein) that specifically inhibits nuclear factor- κB (NF-κB) activity, and administering the chlorite composition to the subject inhibits NF-κB activity by 10% or more, 20% or more, 30% or more, 40% or more, 50% or more, 60% or more, 70% or more, 80% or more, or 90% or more, relative to the level of activity before administering the chlorite composition to the subject.

In certain cases, the subject method is a method for treating acute respiratory distress syndrome (ARDS). Accordingly, as disclosed herein there is provided, a method of treating a subject having or at risk of developing acute respiratory distress syndrome (ARDS), the method comprising: administering a chlorite composition to a subject having or at risk of developing ARDS, wherein the chlorite composition is administered in an amount effective to treat the subject.

In certain cases of the method of treating ARDS, the subject has or is suspected of having a coronavirus infection. In certain cases, the coronavirus is selected from SARS-CoV, MERS-CoV, and SARS-CoV-2. In certain cases, the coronavirus is SARS-CoV. In certain cases, the coronavirus is MERS-CoV. In certain cases, the coronavirus is SARS-CoV-2.

In certain cases of the method of treating ARDS, the subject has or is suspected of having COVID-19. In certain cases, the methods of treating ARDS includes treating a macrophage-associated component of COVID-19.

In certain embodiments, the method is a method of treating a coronavirus infection. Accordingly, as disclosed herein there is also provided a method of treating a subject having or suspected of having a coronavirus infection, the method comprising: administering a chlorite composition to a subject having or suspected of having a coronavirus infection, wherein the chlorite composition is administered in an amount effective to treat the subject.

In certain cases of the method of treating a coronavirus infection, the coronavirus is selected from HCoV-299E, HCoV-OC43, SARS-CoV, HCoV-NL63, HKU1, MERS-CoV, and SARS-CoV-2. In certain cases, the coronavirus is HCoV-299E. In certain cases, the coronavirus is HCoV-OC43. In certain cases, the coronavirus is SARS-CoV. In certain cases, the coronavirus is HCoV-NL63. In certain cases, the coronavirus is HKU1. In certain cases, the coronavirus is MERS-CoV. In certain cases, the coronavirus is SARS-CoV-2.

In certain embodiments of the method of treating a coronavirus infection, the subject has or is at risk of developing ARDS. In certain cases, the subject has been diagnosed, or is suspected of having COVID-19.

In certain embodiments, the method is a method of treating an influenza infection. Accordingly, as disclosed herein there is also provided a method of treating a subject having or suspected of having an influenza infection, the method comprising: administering a chlorite composition to a subject having or suspected of having an influenza infection, wherein the chlorite composition is administered in an amount effective to treat the subject.

In certain embodiments of the method of treating an influenza infection, the subject is infected with an influenza A virus. In certain cases, the influence A virus may be an influenza A group 1 or an influenza A group 2 virus.

### Subjects and Monitoring Therapy

In general, individuals suitable for therapy involving administration of a chlorite composition according to the disclosure include individuals who have been diagnosed as having a macrophage-associated respiratory disorder, are “afflicted with” a macrophage-associated respiratory disorder (e.g., diagnosed as having, suffering from and/or displaying one or more clinical symptoms), or who have been adjudged to be at high risk for developing such a disorder. An “at risk” or “high risk” individual is an individual who has a discrete and significant risk of developing a macrophage-associated respiratory disorder. An “at risk” or “high risk” individual may or may not have detectable disease, and may or may not have displayed detectable disease prior to receiving the method(s) described herein. “High risk” (or “at risk”) denotes that an individual has one or more so-called risk factors, which are measurable parameters that correlate with development of disease. An individual having one or more of these risk factors has a higher probability of developing disease than an individual without these risk factor(s). These risk factors include, but are not limited to, genetic (i.e., hereditary) considerations (including family history and genetic markers). It is understood that having only one risk factor can often indicate high risk. The clinician, as one skilled in the art, has discretion to determine whether treatment using an agent may be indicated for an individual at risk. Example macrophage-associated respiratory disorders includes ARDS, such as ARDS as observed in COVID-19. A macrophage-associated respiratory disorder also includes influenza, e.g., subjects with an influenza A infection.

In one embodiment, individuals suitable for therapy involving administration of a chlorite composition according to the disclosure include individuals who have been diagnosed as having ARDS, are “afflicted with” ARDS (e.g., diagnosed as having, suffering from and/or displaying one or more clinical symptoms of ARDS), or who have been adjudged to be at high risk for developing such a disorder. An “at risk” or “high risk” individual is an individual who has a discrete and significant risk of developing ARDS. An “at risk” or “high risk” individual may or may not have detectable disease, and may or may not have displayed detectable disease prior to receiving the method(s) described herein. “High risk” (or “at risk”) denotes that an individual has one or more so-called risk factors, which are measurable parameters that correlate with development of disease. An individual having one or more of these risk factors has a higher probability of developing disease than an individual without these risk factor(s). These risk factors include, but are not limited to, genetic (i.e., hereditary) considerations (including family history and genetic markers). It is understood that having only one risk factor can often indicate high risk. The clinician, as one skilled in the art, has discretion to determine whether treatment using an agent may be indicated for an individual at risk.

Example clinical symptoms of ARDS include a severe shortness of breath, labored and unusually rapid breathing, low blood pressure, confusion and extreme tiredness. ARDS occurs when fluid builds up in lung alveoli. The fluid prevents the lungs from filling with enough air, limiting the amount of oxygen that reaches the bloodstream which, in turn, deprives the organs of the oxygen they need to function. The symptoms of ARDS can vary in intensity, depending on its cause and severity. In some cases, ARDS is caused by an infected pathogen, such as a respiratory virus, e.g., a coronavirus or influenza.

In another embodiment, individuals suitable for therapy involving administration of a chlorite composition according to the invention include individuals who have been diagnosed as having COVID-19, are “afflicted with” COVID-19 (e.g., diagnosed as having, suffering from and/or displaying one or more clinical symptoms of COVID-19), or who have been adjudged to be at high risk for developing such a disorder. An “at risk” or “high risk” individual is an individual who has a discrete and significant risk of developing COVID-19. An “at risk” or “high risk” individual may or may not have detectable disease, and may or may not have displayed detectable disease prior to receiving the method(s) described herein. “High risk” (or “at risk”) denotes that an individual has one or more so-called risk factors, which are measurable parameters that correlate with development of disease. An individual having one or more of these risk factors has a higher probability of developing disease than an individual without these risk factor(s). These risk factors include, but are not limited to, genetic (i.e., hereditary) considerations (including family history and genetic markers). It is understood that having only one risk factor can often indicate high risk. The clinician, as one skilled in the art, has discretion to determine whether treatment using an agent may be indicated for an individual at risk.

Example clinical symptoms of COVID-19 include those detectable in a biological sample obtained from a subject having or suspected of having COVID-19, e.g., increased levels of one or more inflammatory factors as measured in a biological sample (e.g., blood), such as interleukin-18 (IL-18), C-X-C motif chemokine ligand 9 (CXCL9), C-X- C motif chemokine ligand 10 (CXCL10), interleukin-1 receptor antagonist (IL1Ra), and/or osteopontin (OPN), when compared to native or basal levels in an unaffected biological sample (e.g., blood), or unaffected cells from an unaffected subject. In certain cases, clinical symptoms of COVID-19 include increased levels of one or more soluble plasma factors as measured in a biological sample (e.g., blood), such as C-X-C motif chemokine ligand 9 (CXCL9), C-X-C motif chemokine ligand 10 (CXCL10), interleukin-1 receptor antagonist (IL1Ra), hepatocyte growth factor (HGF), sCD163, osteopontin (OPN), BPI, LBP, S100A8/9 when compared to native or basal levels in an unaffected biological sample (e.g., blood), or unaffected cells from an unaffected subject.

In another embodiment, individuals suitable for therapy involving administration of a chlorite composition according to the disclosure include individuals who have been diagnosed as having influenza, are “afflicted with” influenza (e.g., diagnosed as having, suffering from and/or displaying one or more clinical symptoms of influenza), or who have been adjudged to be at high risk for developing such a disorder. An “at risk” or “high risk” individual is an individual who has a discrete and significant risk of developing influenza. An “at risk” or “high risk” individual may or may not have detectable disease, and may or may not have displayed detectable disease prior to receiving the method(s) described herein. “High risk” (or “at risk”) denotes that an individual has one or more so-called risk factors, which are measurable parameters that correlate with development of disease. An individual having one or more of these risk factors has a higher probability of developing disease than an individual without these risk factor(s). These risk factors include, but are not limited to, genetic (i.e., hereditary) considerations (including family history and genetic markers). It is understood that having only one risk factor can often indicate high risk. The clinician, as one skilled in the art, has discretion to determine whether treatment using an agent may be indicated for an individual at risk.

Example clinical symptoms of influenza include fever, cough, sore throat, runny nose, muscle or body aches, headaches, fatigue, vomiting and diarrhea.

### Microbiome of COVID-19 Patients and Monitoring Therapy

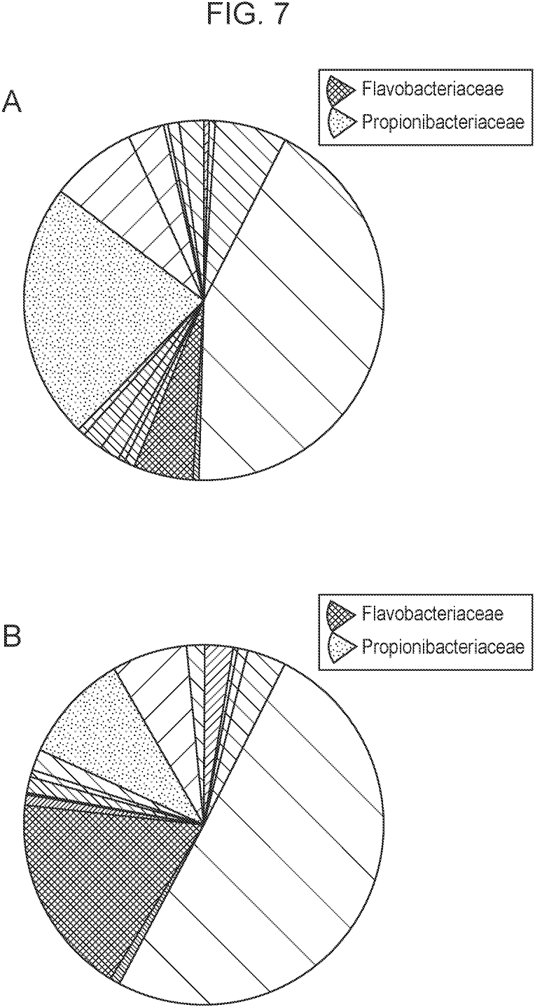

In another embodiment, individuals suitable for therapy may exhibit a microbiome profile representative of a COVID-19 patient. Progress of COVID-19 disease may also be monitored by comparing microbiome profiles of samples derived from the COVID-19 patients. In certain cases, the microbiome is of the nasopharyngeal passage. For example, as seen herein, microbiome profiling of the nasopharyngeal passage derived from 80 COVID-19 patients exhibited different profiles depending on hospital outcome, e.g., alive (discharged patients) vs deceased (see, e.g., FIG. 7, panels A and B respectively, and Example 8). As seen in FIG. 7, panel A, in the profile for COVID-19 patients that were discharged from hospital, the bacteria Flavobacteriaceae were present in an average relative abundance of 5%, as compared to an average relative abundance of 18% in COVID-19 patients who died (FIG. 7, panel B). Further, in the profile for COVID-19 patients that were discharged from hospital, the bacteria Propionibacteriaceae were present in an average relative abundance of 23% (FIG. 7, panel A), as compared to an average relative abundance of 10% in patients who died. Consequently, in some cases, an increase or decrease in a family of bacteria selected from Flavobacteriaceae and Propionibacteriaceae can be used to assess the effect of chlorite-based therapy according to the disclosure herein, e.g., where one or more of a decrease in Flavobacteriaceae bacteria, and an increase in Propionibacteriaceae is indicative of improvement in one or more clinical symptoms in the subject infected with COVID-19.

In certain cases, the microbiome profile is of the gut microbiome. The gut microbiome is an important regulator of immune cell development and function. For example, bacteria can produce short-chain fatty acids, which can bind to innate immune cells and can regulate their metabolism and function and can boost the population of myeloid precursors. In addition, the composition of gut microbiota is known to have important impact on lung immunity and the outcomes of influenza infection. With respect to COVID-19 disease, one study found that severe COVID-19 disease was associated with decreased abundance of butyrate-producing bacteria and increased numbers of common opportunistic pathogens (e.g. Enterococcus, Enterobacteriaceae) in the gut (Tang L, Gu S, Gong Y, Bo Li HL, Li Q, Zhang R, et al. Clinical Significance of the Correlation between Changes in the Major Intestinal Bacteria Species and COVID-19 Severity. Engineering. 2020) while another study found decreased diversity with higher numbers of opportunistic pathogens (Gu S, Chen Y, Wu Z, Gao H, Lv L, Guo F, et al. Alterations of the Gut Microbiota in Patients with COVID-19 or H1N1 Influenza. Clin Infect Dis. 2020). Furthermore, certain bacteria (e.g. Lactobacillus and Bifidobacterium) are known to play an important role in maintaining intestinal barrier function. Without being bound to any particular theory, alterations of these commensal species could disrupt the intestinal barrier leading to microbial translocation, whereby microbes that are normally restricted in the gut are now present in the blood. Accordingly, monitoring of the presence and levels of these commensal species can be used to assess the effect of chlorite-based therapy according to the disclosure herein, e.g., such as increased abundance of bacteria such as Lactobacillus and Bifidobacterium, and decreased numbers of common opportunistic pathogens being indicative of improvement in one or more clinical symptoms in the subject infected with COVID-19.

Microbial translocation can be associated with inflammation and COVID-19 disease. Chronic immune activation is frequently associated with microbial translocation. Microbial translocation results in high levels of lipopolysaccharide (LPS), a component of gram-negative bacterial cell membrane, which is a potent macrophage activator and can contribute to the chronic pathogenic inflammation associated with many neurodegenerative and immune dysfunction diseases. A recent study suggested that ~50% of COVID-19 patients requiring ICU support showed evidence for microbial translocation, with bacterial products in the blood contributing to the most severe forms of immune dysfunction (Sirivongrangson. Endotoxemia and circulating bacteriome in severe COVID-19 patients. pre-print, medRxiv. 2020).

Accordingly, the blood microbiome can be a marker for gut function, and as such, distinct blood microbiomes can be characterized and associated with COVID-19 to monitor disease progression.

### Monitoring Therapy

Chlorite-based therapy according to the invention can be monitored, and dosages and regimen adjusted accordingly, by assessing the effect of therapy upon one or more clinical symptoms. In general, an effective amount of chlorite is a dose or doses that provide for an improvement in one or more clinical symptoms in the subject.

Methods for assessing elevated levels of factors associated with COVID-19 patients through assessment of biological samples is described in, for example, in Blanco-Melo et al., Cell (2020), 181(5), 1036-1045, (also referred to herein as “the Blanco-Melo study”); Chen et al. 2020, Frontiers in Microbiology 10:50, 1-9; and Li et al. 2020, Journal of Pharmaceutical Analysis, 10: 102-108.

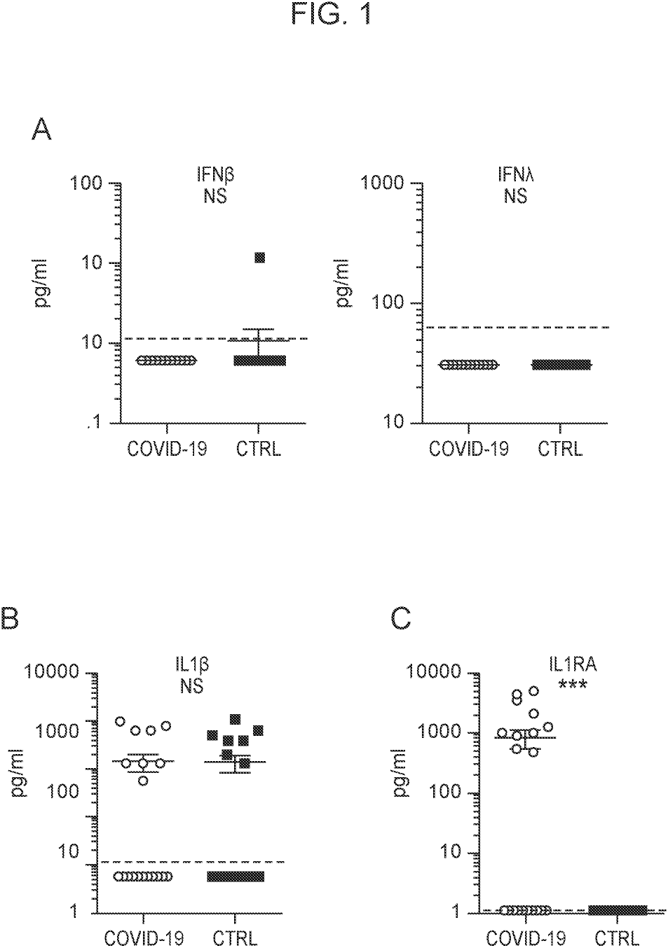

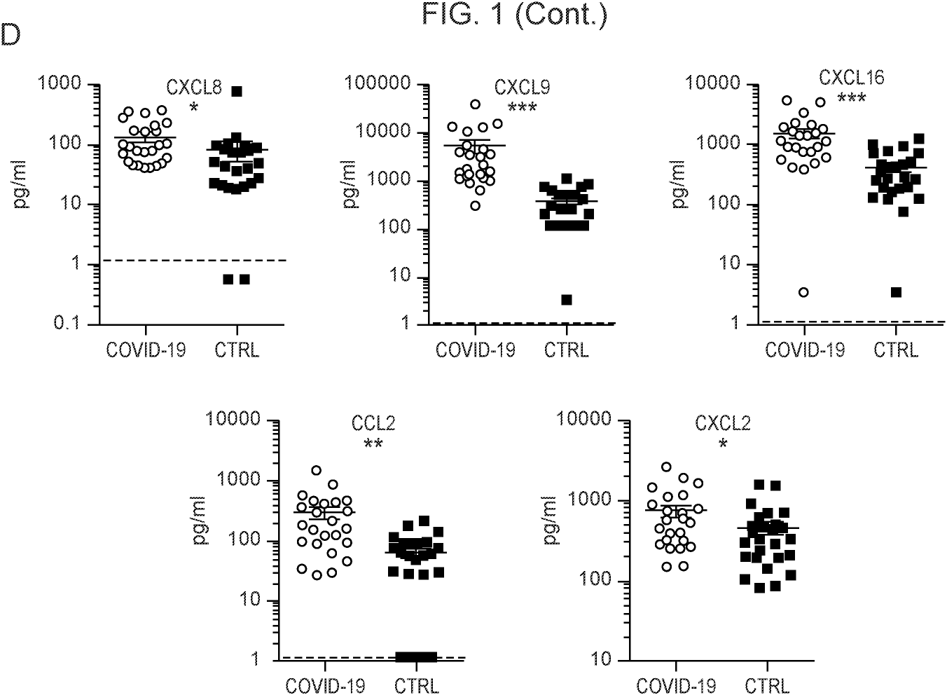

For example, therapy for a macrophage-associated respiratory disorder, such as ARDS, e.g., as can be associated withCOVID-19 therapy can be monitored by determining the plasma levels of factors associated with COVID-19 “cytokine storm” (see, e.g., FIG. 1, “the Blanco-Melo study” results). In certain cases, the plasma sample has elevated levels of one or more inflammatory factors selected from one or more of interleukin-18 (IL-18), C-X-C motif chemokine ligand 9 (CXCL9), C-X-C motif chemokine ligand 10 (CXCL10), and interleukin-1 receptor antagonist (IL1Ra). In certain cases, the plasma sample has elevated levels of one or more soluble plasma factors selected from one or more of C-X-C motif chemokine ligand 9 (CXCL9), C-X-C motif chemokine ligand 10 (CXCL10), interleukin-1 receptor antagonist (IL1Ra), hepatocyte growth factor (HGF), sCD163, osteopontin (OPN), BPI, LBP, and S100A8/9. As such, the method includes the step of determining the level of one or more of interleukin-18 (IL-18), C-X-C motif chemokine ligand 9 (CXCL9), C-X-C motif chemokine ligand 10 (CXCL10), osteopontin (OPN), interleukin-1 receptor antagonist (IL1Ra), hepatocyte growth factor (HGF), sCD163, BPI, LBP, and S100A8/9 in a plasma sample from a patient having or suspected of having a macrophage-associated respiratory disorder, such as ARDS, e.g., as can be associated with COVID-19. In another embodiment, the level of one or more of interleukin-18 (IL-18), C-X-C motif chemokine ligand 9 (CXCL9), C-X-C motif chemokine ligand 10 (CXCL10), osteopontin (OPN), interleukin-1 receptor antagonist (IL1Ra), hepatocyte growth factor (HGF), sCD163, BPI, LBP, and S100A8/9 in a plasma sample are determined during and/or at completion of the therapy, and is generally compared with the level in a control sample and/or with a desired value.

Accordingly, there is provided a method of determining inhibition of an acute respiratory distress syndrome (ARDS)-induced macrophage activity by a chlorite agent, comprising: contacting a macrophage of a subject having ARDS with a chlorite agent; and assessing a level of one or more inflammatory factors selected from interleukin-18 (IL-18), C-X-C motif chemokine ligand 9 (CXCL9), C-X-C motif chemokine ligand 10 (CXCL10), osteopontin (OPN), and interleukin-1 receptor antagonist (IL1Ra) after said contacting, wherein a decrease in the level of the inflammatory factor after said contacting indicates that the chlorite agent is an inhibitor of the ARDS-induced macrophage activity. There is also provided a method of determining inhibition of an acute respiratory distress syndrome (ARDS)-induced macrophage activity by a chlorite agent, comprising: contacting a macrophage of a subject having ARDS with a chlorite agent; and assessing a level of one or more soluble plasma factors selected from one or more of C-X-C motif chemokine ligand 9 (CXCL9), C-X-C motif chemokine ligand 10 (CXCL10), interleukin-1 receptor antagonist (IL1Ra), hepatocyte growth factor (HGF), sCD163, osteopontin (OPN), BPI, LBP, and S100A8/9 after said contacting, wherein a decrease in the level of the soluble plasma factor after said contacting indicates that the chlorite agent is an inhibitor of the ARDS-induced macrophage activity. There is further provided a method of determining inhibition of an acute respiratory distress syndrome (ARDS)-induced macrophage activity by a chlorite agent, comprising: contacting a macrophage of a subject having ARDS with a chlorite agent; and assessing a level of nuclear factor- κB (NF-κB) activity, where a decrease in activity after said contacting indicates that the chlorite agent is an inhibitor of the ARDS-induced macrophage activity.

In certain cases, the method of determining inhibition of an ARDS-induced macrophage activity by a chlorite agent includes measuring the level of the one or more inflammatory factors (e.g., as described herein) prior to said contacting. In certain cases, the method of determining inhibition of an ARDS-induced macrophage activity by a chlorite agent includes measuring the level of the one or more soluble plasma factors (e.g., as described herein) prior to said contacting. In certain cases, the method of determining inhibition of an ARDS-induced macrophage activity by a chlorite agent includes measuring the level of nuclear factor- κB (NF-κB) prior to said contacting. In certain cases of the method of determining inhibition of an ARDS-induced macrophage activity by a chlorite agent the assessing step is at least one day after said contacting, such as 1 day, 2 days, 3 days, 4 days, 5 days, 6 days, or 7 days after said contacting.

In certain cases of the method of determining inhibition of an ARDS-induced macrophage activity by a chlorite agent, the factor assessed is C-X-C motif chemokine ligand 9 (CXCL9). In certain cases of the method of determining inhibition of an ARDS-induced macrophage activity by a chlorite agent, the factor assessed is interleukin-1 receptor antagonist (IL1Ra). In certain cases of the method of determining inhibition of an ARDS-induced macrophage activity by a chlorite agent, the factor assessed is C-X-C motif chemokine ligand 10 (CXCL10). In certain cases of the method of determining inhibition of an ARDS-induced macrophage activity by a chlorite agent, the factor assessed is osteopontin (OPN). In certain cases of the method of determining inhibition of an ARDS-induced macrophage activity by a chlorite agent, the factor assessed is hepatocyte growth factor (HGF). In certain cases of the method of determining inhibition of an ARDS-induced macrophage activity by a chlorite agent, the factor assessed is sCD163. In certain cases of the method of determining inhibition of an ARDS-induced macrophage activity by a chlorite agent, the factor assessed is BPI. In certain cases of the method of determining inhibition of an ARDS-induced macrophage activity by a chlorite agent, the factor assessed is LBP. In certain cases of the method of determining inhibition of an ARDS-induced macrophage activity by a chlorite agent, the factor assessed is S100A8/9.

Further, since elevated HLA-DR expression on CD14+ cells and/or increased numbers of CD14+/CD16+ cells and/or the percentage of CD16+ cells in a population of CD14+ cells is associated with a macrophage-associated disorder, monitoring these levels can be used to facilitates assessment of initial responsiveness to therapy and/or efficacy, as well as the appropriate dosage of the therapy.

It is understood that monitoring therapy means that symptoms are assessed at different times and are compared over time. Where assessment of a clinical symptom requires analysis of a biological sample, such biological sample(s) are generally obtained at different times, for example, during application of therapy, and are compared, either with each other, a control, and/or a desired value.

For example, therapy for a macrophage-associated respiratory disorder, such as ARDS, e.g., as can be associated with COVID-19, can be monitored by determining the level of expression by CD14+ cells from peripheral blood. In another embodiment, monitoring therapy includes the step of determining the level of CD14+ cells expressing elevated HLA-DR in a blood sample, such as peripheral blood. In another embodiment, monitoring therapy includes the step of determining the percentage of CD16+ cells in the population of CD14+ cells in a blood sample, such as peripheral blood. In another embodiment, monitoring therapy includes the step of determining the number of CD14+/CD16+ cells in a blood sample, such as peripheral blood. In another embodiment, the level of abnormal macrophages (in various embodiments, the level of CD14+ cells expressing elevated HLA-DR; the percentage of CD16+ cells in the population of CD14+ cells and/or the number of CD14+/CD16+ cells) in a blood sample determined during and/or at completion of the therapy is generally compared with the level in a control sample and/or with a desired value. In another embodiment, monitoring therapy also includes the step of measuring proliferation of the abnormal macrophages.

In another embodiment, therapy for a macrophage-associated respiratory disorder, such as ARDS, e.g., as can be associated with COVID-19, is monitored by assessing the level of abnormal macrophages in a sample taken at a particular time from a patient undergoing the therapy and/or a sample taken after or at completion of the therapy is generally compared with the level in a sample taken from the patient prior to the therapy and/or with the level in a sample taken from the patient at a different time point in the therapy. For example, a decrease in the level of abnormal macrophages in the sample taken during therapy as compared to the sample taken prior to or at an earlier time point in therapy would generally be consistent with a positive effect of the therapy.

In another embodiment, therapy according to the invention is monitored by assessing the level of abnormal macrophages is assessed by the determining the level of HLA-DR expression by CD14+ cells from a blood sample, such as a peripheral blood sample. For example, the effect of a therapy is determined by comparing the level of HLA-DR expression by CD14+ cells in peripheral blood before and during treatment, with a downward trend in HLA-DR expression generally being consistent with a positive effect.

In another embodiment, therapy according to the invention is monitored by assessing the level of pathologic macrophages, e.g., by assessing the level of abnormal macrophages is assessed by the determining the percentage of CD16+ cells in the population of CD14+ cells from a blood sample, such as a peripheral blood sample. For example, the effect of a therapy is determined by comparing the percentage of CD16+ cells in the population of CD14+ cells in peripheral blood before and during treatment, with a downward trend in the percentage of CD14+/CD16+ cells generally being consistent with a positive effect.

In another embodiment, therapy according to the invention is monitored by assessing the level of pathologic macrophages, e.g., by assessing the level of abnormal macrophages is assessed by the determining the number of CD14+/CD16+ cells in a blood sample, such as a peripheral blood sample. For example, the effect of a therapy is determined by comparing the number of CD14+/CD 16+ cells in peripheral blood before and during treatment, with a downward trend in the number of CD14+/CD16+ cells generally being consistent with a positive effect.

In another embodiment, therapy according to the invention is monitored by assessing the presence and level of bacterial 16S DNA and soluble bacterial by-products e.g., by assessing the presence and or level of bacterial 16S DNA, and soluble bacterial by-products in a blood sample from the patient, such as a peripheral blood sample. For example, the effect of a therapy is determined by comparing the level of bacterial 16S DNA in peripheral blood before and during treatment, with a downward trend in bacterial 16S DNA generally being consistent with a positive effect.

### Kits

The invention also contemplates kits with unit doses of a source of chlorite ions, e.g., a chlorite composition as described herein above. In general such unit doses are in injectable dosage forms, more particularly dosage forms suitable for infusion. In such kits, in addition to the containers containing the unit doses will be an informational package insert describing the use and attendant benefits of chlorite in treating a macrophage-associated respiratory disorder subject, such as ARDS, e.g., as can be associated with COVID-19. Optionally, the kit includes information relating to identification of patients having a macrophage-associated respiratory disease and monitoring of therapy of such patients (e.g., information relating to assessment of pathologic macrophages, e.g.,. proliferating macrophages, activated macrophages, and information relating to measuring levels of relevant factors such as IL-18, CXCL9, CXCL10, IL1Ra, osteopontin (OPN), sCD163, hepatocyte growth factor (HGF), bactericidal/permeability-increasing protein (BPI), lipopolysaccharide binding protein (LBP), Calprotectin (S100A8/9), sCD14 and nuclear factor- κB (NF-κB).

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

General methods in molecular and cellular biochemistry can be found in such standard textbooks as Molecular Cloning: A Laboratory Manual, 3rd Ed. (Sambrook et al., HaRBor Laboratory Press 2001); Short Protocols in Molecular Biology, 4th Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Lefkovits ed., Academic Press 1997); and Cell and Tissue Culture: Laboratory Procedures in Biotechnology (Doyle & Griffiths, John Wiley & Sons 1998), the disclosures of which are incorporated herein by reference. Reagents, cloning vectors, cells, and kits for methods referred to in, or related to, this disclosure are available from commercial vendors such as BioRad, Agilent Technologies, Thermo Fisher Scientific, Sigma-Aldrich, New England Biolabs (NEB), Takara Bio USA, Inc., and the like, as well as repositories such as e.g., Addgene, Inc., American Type Culture Collection (ATCC), and the like.

### Introduction

Human infection with SARS-Cov-2 leads to two general clinical outcomes based on the pattern of disease pathogenesis. As the virus is transmitted as an aerosolized pathogen, the first cells to become infected are the upper airway respiratory epithelial cells. As in other classes of upper airway viral infections, this type of infection with SARS-Cov-2 can result in a normal T cell mediated immune response leading to the clearance of the infection. This first pattern of disease more often affects generally healthy, younger individuals and generally does not require oxygen therapy or hospitalization for treatment. The second pattern of COVID-19 disease is more severe, and often affects those who are older, and/or have intercurrent illnesses such as diabetes, cardiac disease, dementia or obesity. These are the patients who require oxygen support, hospitalization and in very severe disease respiratory ventilation. The first pattern of COVID-19 is a relatively severe conventional upper airway epithelial cell viral infection with normal immune system clearance within 1-2 weeks; the second pattern involves chronic infection of tissue based lung and other organ macrophages, a process that can result in a self-perpetuating “cytokine storm” wherein tissue macrophages elaborate factors that lead to persistence of a type of destructive tissue based inflammation.

The second pattern referred to above, and most deadly form of COVID-19 was recently reviewed (Li et al., Journal of Pharmaceutical Analysis, (2020), 10, 102-108). It is believed that through inhibition of the interferon (anti-viral) immune response and induction of chemokines and factors that recruit inflammatory cells to diseased microenvironments, SARS-Cov-2 infected cells can avoid the immune response and drive a perpetual cytokine storm. In has been proposed that several of the SARS-Cov-2 encoded proteins can either directly activate a form of inflammation termed pyroptosis through inflammasome induction (Chen et al., Frontiers in Microbiology (2020), 10:50, 1-9) leading to elevation of IL18, or through blocking of interferon induction by the normal infected cell (Li et al., Journal of Pharmaceutical Analysis, (2020), 10, 102-108). A more recent study of autopsy tissues with parallel quantitative gene expression of tissue RNAs paired with plasma ELISA evaluation of factors elevated in severe COVID-19 disease was recently published (Blanco-Melo et al., Cell (2020), 181(5), 1036-1045, referred to herein as “the Blanco-Melo study”).

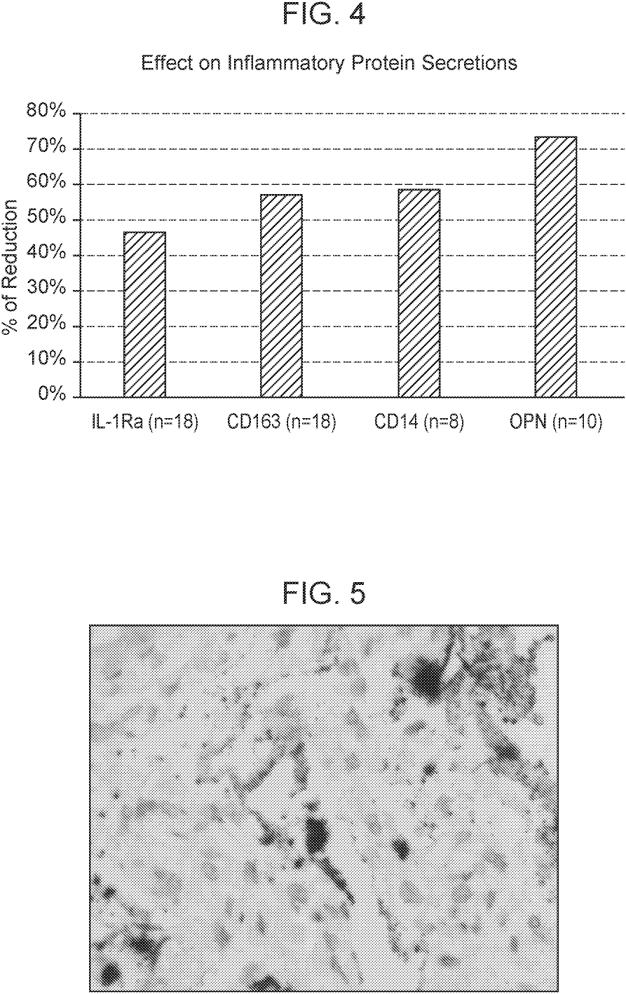

The Blanco-Melo study (see, e.g., Blanco-Melo et al., Cell (2020), 181(5), 1036-1045, FIG. 4) suggests various factor levels of cytokines, chemokines and interleukins that can define the severe form of COVID-19 disease, and this is summarized in FIG. 1 herein.

Accordingly, based on the Blanco-Melo study FIG. 1, panels A to D illustrate plasma levels of factors associated with COVID-19 “cytokine storm” are significantly elevated in plasma of severe COVID-19 patients as compared to a control. Panel A shows factors that should be elevated in normal antiviral response, are not elevated in severe COVID-19 patients. Specifically, levels of IFNβ (upper), and IFNλ (lower) exhibit no significant difference between severe COVID-19 patients and their respective controls. Panel B shows levels of IL1β in severe COVID-19 patients and a control. IL1β is required for immune response initiation. As shown in panel B, there is no significant difference between the severe COVID-19 patients and the control, indicating that IL1β is not elevated in severe COVID-19 patients. Panel C shows levels of IL1RA in severe COVID-19 patients and control. IL1 receptor antagonist (IL1RA) levels are high in severe COVID-19 patients, thus blocking IL1 function and immune response. Panel D illustrates chemokines that attract monocytes and macrophages into chronic inflammatory environment perpetuating severe COVID-19 disease. As shown in panel D, it was observed in the Blanco-Melo study that the chemokine levels for factors attracting macrophages and granulocytes into diseased tissues were all significantly elevated in severe COVID-19 patients as compared to their respective controls.

### General Methods

### Sample Collection

Over the course of a 2-year study 30 heparinized blood samples (2 vials per patient) are collected derived from patients with moderate to severe COVID-19 infections. samples will be immediately shipped to the laboratory. Limited de-identified patient clinical information linked to samples will be provided, as approved under a standing IRB (patient age, disease severity, comorbidities, Ct values).

### Whole Blood and Blood Fractions Sample Preparation

Two heparinized blood tubes (4 mL each per donor) are collected and cold shipped to the laboratory. One blood tube from each donor is aliquoted (500µL) under a Class II biologic safety cabinet (BSCII) and stored at -80° C. until shipment or DNA extraction. The other blood tube from each donor is used for plasma, buffy coat (PBMC), and red blood cell (RBC) separation using Ficoll Paque PLUS prepared in BSCII. After centrifugation, plasma is aliquoted (500µL, 3-4 aliquots), and immediately frozen at -80° C. until shipment to core immunology facility on dry ice. The PBMC phase is carefully collected, washed and counted. For 15 selected cases for the studies described herein, viable cell suspension of ~1 × 10⁶ cells/mL using RPMI-1640 complete cell culture media is made in triplicate in polypropylene culture tubes for immediate chlorite composition or control treatment. All remaining PBMCs are stored in freezing medium at -80° C.

### DNA and RNA Extraction

DNA from PBMCs is isolated in a BSCII using a stringent contamination-aware approach previously described (Païssé S, Valle C, Servant F, Courtney M, Burcelin R, Amar J, et al. Comprehensive description of blood microbiome from healthy donors assessed by 16S targeted metagenomic sequencing. Transfusion. 2016;56(5):1138-47) using a DNA isolation kit NucleoSpin blood kit,(Macherey-Nagel). The DNA quality is monitored by gel electrophoresis and quantity is measured using an Invitrogen Qubit 3 assay. All DNA isolations are performed in triplicate. RNA is isolated from plasma using the Zymo Quick Viral RNA kit and the maximum sample volume (400µL), with quantification performed on the Qubit.

### Quantification of SARS-CoV-2 Using ddPCR

Plasma RNA is assayed using the BIORAD EUA SARS-CoV-2 assay on the Biorad QX200 droplet digital PCR machine to calculate viral copy number in each blood sample. This triple assay simultaneously measures the nucleocapsid gene with the CDC N1 and N2 targets, as well as the human RNAseP gene. The ddPCR system is highly sensitive, and does not require standards to calculate copy number, increasing comparability of results over the course of the study.

### 16S Copy Number Using ddPCR

The 16S copy number in DNA from plasma and PBMC is quantified by droplet digital PCR (ddPCR) targeting the V3-V4 hypervariable regions of the bacterial 16S rRNA gene with universal primers EUBF and EUBR (46), which have been extensively tested to determine that no eukaryotic or mitochondrial DNA will be amplified as well (Païssé S, Valle C, Servant F, Courtney M, Burcelin R, Amar J, et al. Comprehensive description of blood microbiome from healthy donors assessed by 16S targeted metagenomic sequencing. Transfusion. 2016;56(5):1138-47). The results are reported as 16S rRNA gene copies per ng of total DNA and per µl of blood. All reagents are tested with ddPCR to quantify impact of bacterial contaminants.

### Endotoxin Measurements

The chemiluminescent-based endotoxin activity assay (EAA; Spectral Diagnostics, Ontario, Canada) is performed as described elsewhere (Romaschin AD, Harris DM, Ribeiro MB, Paice J, Foster DM, Walker PM, et al. A rapid assay of endotoxin in whole blood using autologous neutrophil dependent chemiluminescence. J Immunol Methods. 1998;212(2):169-85). This is a detection assay of enhanced respiratory burst activity in neutrophils following their priming by complexes of endotoxin and a specific anti-endotoxin antibody. Briefly, 40 µL of whole blood is incubated with zymosan and anti-endotoxin antibody. The endotoxin activity level of 0.60 is considered as high activity level.

### Inflammatory Markers

Using the frozen plasma, the following soluble plasma factors associated with cellular activation, inflammation and infiltration of bacteria are simultaneously evaluated on the Luminex MAGPIX system: IFN-y, IL-1Ra, IL-2Ra, IL-6, IL-10, IL-18, HGF, MCP-3, MIG (CXCL9), M-CSF, G-CSF, MIP-1a, IP-10 (CXCL10), LPS-binding protein (LBP), BPI, sCD14, sCD163, Osteopontin, Calprotectin (S100A8/9), MCP-1.

### Sodium Chlorite in Vitro PBMC Cultures

PBMC suspensions are subjected to treatment with either 300 µM of a sodium chlorite composition (e.g., as disclosed herein) or control. Freshly prepared working dilutions of the sodium chlorite composition are prepared immediately prior to the experiment. 100 µL of working dilution is added into 900 µL of PBMC suspension to achieve the final concentration of 300 µM. Cultures are incubated in a humidified incubator at 37° C. with a 5% CO₂ atmosphere for 3 days. After incubation, PBMCs are pelleted and counted, and supernatants collected, with both stored at -80° C. Triplicate supernatants for cytokine assays from treated and control experiments, as well as PBMCs for single cell assays, are prepared.

### Single Cell Transcriptomics and ELISAs

Control and treated PBMCs are loaded on the 10X Chromium controller to assess the single cell transcriptional profile. Single cell ELISAs are performed on the Isoplexis system. For the single cell ELISAs, cultured PBMCs are enriched for myeloid cells using CD33 magnetic beads. The enriched cells are loaded onto the IsoLight Single Cell Innate chip where the automated process of measuring the 30+ cytokines and chemokines occurs.

### Example 1- Sodium Chlorite Regulates Macrophage Activation and Production of Activation Associate Cytokine Genes And Factors in Vitro

Normal blood derived macrophages were cultivated for 3 days in the presence of increasing amounts of an example sodium chlorite composition (greater than 99% purity, formulated for intravenous administration). Cells were then processed for flow cytometry (5 separate donors) and CD14 cell co-expression of CD16 (macrophage activation marker) was scored.

Specifically, the sodium chlorite formulation was tested in 5 blood cell preparations at increasing concentration (e.g., +30 µM; +100 µM; and +300 µM) for three days. All CD14 cells become CD16 bright in vitro. Medium CD16 levels on CD14 cells were determined after three days of treatment for control untreated (normalized to 100%), and with increasing levels of sodium chlorite. Percentage of control median CD16 was calculated for all 5 donors +/- SD.

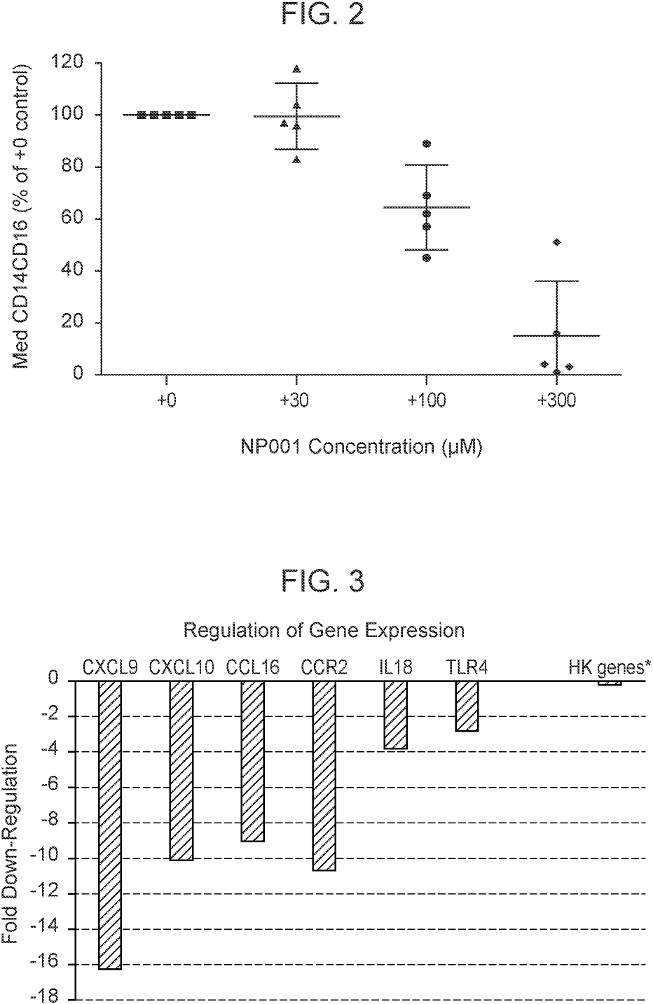

FIG. 2 shows that the subject sodium chlorite formulation regulates in vitro CD16 expression on amyotrophic lateral sclerosis (ALS) macrophages; and a dose dependent down regulation of CD16 on CD14+ (monocyte lineage) cells. The data in FIG. 2 indicates that sodium chlorite (e.g., in a formulation as described herein) regulates macrophage activation in vitro in a dose dependent manner.

### Example 2 - Sodium Chlorite Effect on Gene Regulation

Normal blood macrophages from three donors were treated for three days with an example sodium chlorite composition (greater than 99% purity, formulated for intravenous administration) (150uM), as in Example 1 and FIG. 2, then RNA was converted into cDNA and applied to an Affymetrix Human genome U133 plus gene chip and selected expressed genes were scored for being responsive to sodium chlorite.

### Macrophage Genes Down Regulated> 2x

1) CXCL9: 14X
2) CCR3: 3X

The gene observed to be most regulated by the sodium chlorite formulation in normal macrophages was CXCL9, also known as macrophage chemokine induced by gamma interferon (MIG). CXCL9 overexpression is associated with ongoing inflammation and migration of monocytes into diseased tissues. Down regulation of this molecule can be associated with resolution of an inflammatory process.

### Macrophage Genes Upregulated> 2x

1) SERPINB2: 4X

Upregulation of macrophage SERPINB2 is associated with inhibition of apoptosis and resolution of immune response/inflammation.

### Example 3 - Sodium Chlorite Effect on Inflammatory Gene Expression

An example sodium chlorite composition (greater than 99% purity, formulated for intravenous administration) was observed to regulate macrophage genes to a greater degree in macrophages from diseased patients as compared to normal macrophages. Blood derived macrophages from two ALS patients whose blood monocytes showed elevation of CD14 cell HLA-DR (Zhang et al. J Neuroimmunol. 2005 Feb;159(1-2):215-24. Epub 2004 Nov 26) consistent with disease associated inflammation were treated with the sodium chlorite formulation (300uM) overnight prior to evaluation by the UCSF molecular core gene expression lab using a panel of genes using Illumina based technology. The maj or focus was on regulation of inflammation as compared to normal macrophage functional genes. FIG. 3 shows the effect of the sodium chlorite formulation on inflammatory gene expression. More particularly, FIG. 3 shows clear down regulation of genes related to inflammation maintenance (CXCL9, CXCL10, CCL16, CCR2, and TLR4) and inflammasome activation (IL-18). There was no cell death and the housekeeping gene levels remained unchanged from untreated controls. Note that by “housekeeping genes” is meant B2M, HPRT1, RPL13A, GAPDH, and ACTB.

As such, the subject sodium chlorite formulation exhibits more gene expression regulatory activity in monocytes/macrophages obtained from patients with macrophage associated disease (ALS) than in regulation of normal cell function.

### Example 4 - Als Blood Macrophage Secretion of Inflammatory Factors Is Inhibited by Sodium Chlorite

ALS blood monocytes were cultured for 3 days with 300uM of an example sodium chlorite composition (greater than 99% purity, formulated for intravenous administration) as in FIGS. 2 and 3. Culture supernatants were harvested and cytokine levels were determined by ELISA. FIG. 4 illustrates that the subject sodium chlorite formulation inhibits production of secreted inflammatory factors from ALS patient macrophages. Referring to the Y-axis, percentage of reduction is the level of factor in treated as compared to untreated macrophages. N is 8-18 in several experiments. IL-1Ra is IL-1 receptor antagonist, a factor that inhibits normal immune response. CD163 is secreted by activated macrophages as they migrate into tissues. CD14 is the LPS receptor and lower levels are associated with less macrophage activation. Osteopontin (OPN) is a potent monocyte chemotactic factor causing migration of monocytes from blood into sites of chronic inflammation, thus maintaining a chronic inflammatory state.

It was observed that sodium chlorite treatment was non-toxic and induced ALS macrophages to down regulate production of factors critical to maintenance of a chronic inflammatory environment.

### Example 5 - Reversal of Production of Pathogenic Levels of Factors Associated With Covid-19

Severe COVID 19 disease is characterized by chronic macrophage activation with production of factors as outlined in FIG. 1. As shown above many of the factors shown in FIG. 1 are regulated by an example chlorite composition (e.g., as described herein) at the level of gene expression. Accordingly, it was sought to investigate if an example sodium chlorite composition (greater than 99% purity, formulated for intravenous administration) could reverse the production of pathogenic levels of factors such as those described in the Blanco-Melo study, summarized in FIG. 1 in COVID-19 patients.

To investigate this hypothesis, heparinized blood was isolated from 6 outpatients with COVID-19. Plasma levels of cytokine storm factors related to COVID-19 disease activity that were 5 times or more higher than normal (e.g., 5 to 700 times higher than normal) are as follows:
Macrophage cytokine storm factors: interferon gamma-induced protein 10 (IP10, also referred to herein as CXCL10), monokine induced by gamma interferon (MIG, also referred to herein as CXCL9), macrophage inflammatory protein 1-alpha MIP1a (also known as chemokine (C-C motif) ligand 3 (CCL3)), IL1Ra, interferon gamma (IFNγ), Hepatocyte Growth Factor (HGF), and sCD163 (also referred to herein is CD163); and
COVID-19 microbial translocation and monocyte trafficking associated factors: bactericidal/permeability-increasing protein (BPI), lipopolysaccharide binding protein (LBP), and S100 calcium-binding proteins A8 and A9 (S100A8 and S100A9).

Peripheral blood mononuclear cell (PBMC) cultures from COVID-19 patients was cultured for 3 days with sodium chlorite composition (greater than 99% purity, formulated for intravenous administration).

COVID19 blood monocytes were cultured for 3 days with a dose equivalent to 1 mg/kg of an example sodium chlorite composition (greater than 99% purity, formulated for intravenous administration) diluted into normal blood volume (see e.g., procedure outlined under General Methods). Culture supernatants were harvested and cytokine levels were determined by ELISA. The following COVID-19 macrophage produced factors were measured after sodium chlorite treatment, and compared to the control levels:
Macrophage cytokine storm factors-IL-1ra, IL-6, IL-10, G-CSF, IP-10 (CXCL10), IFN-γ, MCP-1, MIP-1a, IL-2ra, IL-18, HGF, MCP-3, M-CSF, MIG (CXCL9), OPN.

Microbial translocation and monocyte trafficking- bactericidal/permeability-increasing protein (BPI), LPS binding protein (LBP), Calprotectin (S100A8/9), sCD14, sCD163

The subject sodium chlorite formulation down regulated the following factors by greater than 50%: IP10 (CXCL10), MIG (CXCL9), OPN, IL1Ra, HGF, OPN and sCD163 (CD163). As noted above, the following factors are elevated in COVID-19 plasma by 5 times or more: IP10 (CXCL9), IL-1Ra, BPI, LBP, sCD163, S100A8/9.

Table 1 shows the effects of the subject sodium chlorite formulation (“chlorite”) on reducing the level the factors IP10 (CXCL10) and MIG (CXCL9) in vitro in mild and moderate COVID-19 plasma, as compared to control plasma levels, e.g., in normal plasma, mild COVID-19 plasma, and moderate COVID-19 plasma.

TABLE 1

| Plasma IP10 | Plasma level (pg/mL) | Chlorite % reduction of factor in vitro | Plasma MIG | Plasma level (pg/mL) | Chlorite % Reduction of factor in vitro |
|---|---|---|---|---|---|
| Normal Plasma | 105 | | Normal Plasma | 56 | |
| Mild COVID-19 Plasma | 625 | 95% | Mild COVID-19 Plasma | 660 | 88% |
| Moderate COVID-19 Plasma | 2700 | 80% | Moderate COVID-19 Plasma | 2000 | 70% |

As demonstrated above in Table 1, the subject chlorite formulation dramatically reduces the level of the factors IP 10 (CXCL10) and MIG (CXCL9) in blood plasma from both mild and moderate COVID-19 patients.

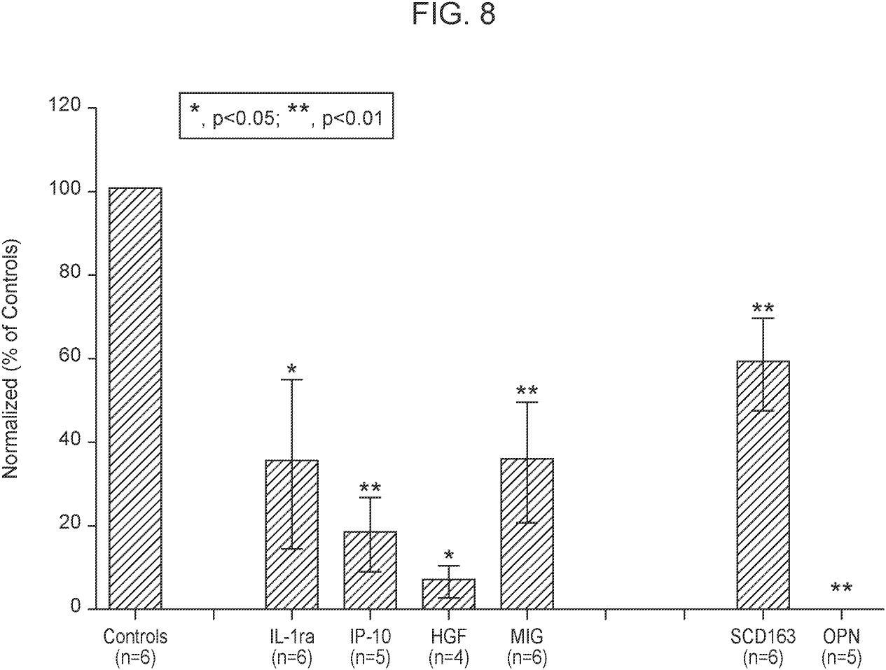

FIG. 8 further illustrates that the subject sodium chlorite formulation downregulated production of COVID-19 patient macrophage produced disease factors as compared to controls. Referring to the Y-axis, percentage of controls is the level of factor in treated as compared to the control or untreated macrophages. IL-1Ra, IP-10 (CXCL10), HGF, and MIG (CXCL9) are factors related to cytokine storm. sCD163 (CD163) and OPN are factors related to monocyte traffic to tissues and persistent activation.

In summary sodium chlorite treatment is non-toxic and as demonstrated herein can induce COVID-19 macrophages to down regulate production of factors critical to maintenance of a chronic inflammatory environment.

### Example 6- Monocyte Associated Osteopontin (Opn) Is Inhibited by in Vitro Treatment With Sodium Chlorite

ALS blood monocytes (one million) are cultured for 3 days with 150uM of an example sodium chlorite composition (greater than 99% purity, formulated for intravenous administration) as outlined in Example 4. Culture supernatants are harvested and osteopontin levels (OPN) are determined by ELISA.

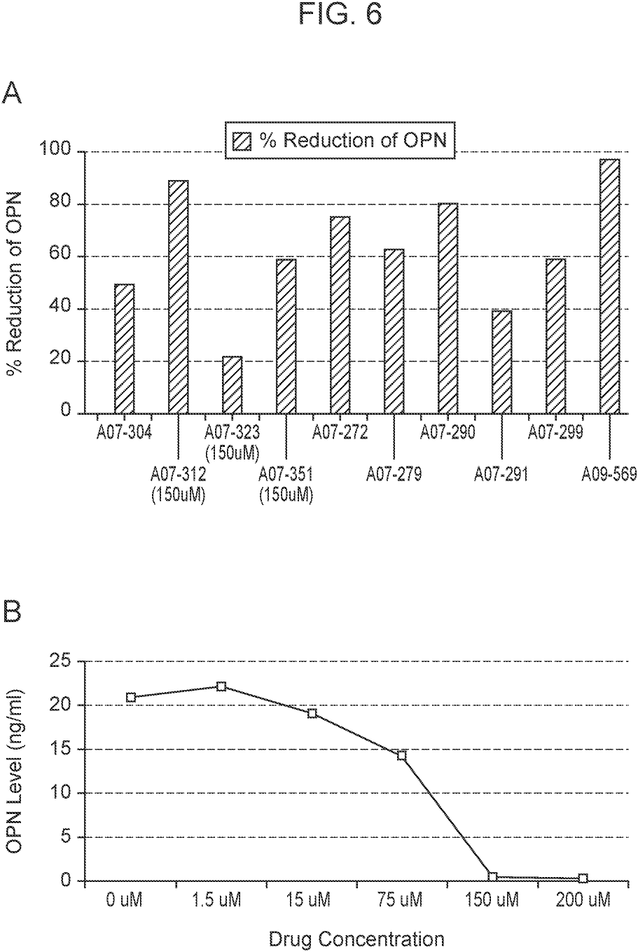

FIG. 6, panel A shows that the subject sodium chlorite formulation can inhibit production of OPN from ALS patient macrophages. FIG. 6, panel B shows quantitative SPP1 (osteopontin) gene expression in ALS blood monocytes cultured for 3 days with 150 uM of an example sodium chlorite composition as a dose response. As seen in panel B, monocyte produced OPN is inhibited by culture with the subject sodium chlorite formulation over 3 days. Note that RT-PCR for OPN RNA shows same level of regulation. Repeated with 3 normal and 5 ALS donors.

Sodium chlorite treatment is can induce ALS macrophages to down regulate production of OPN.

### Example 7 - Sodium Chlorite Treatment of Covid-19 Patients Is Associated With Down Regulation of Systemic Factors Associated With Ongoing Inflammatory Disease Activity

A safety, tolerability and pharmacodynamic marker evaluation study is undertaken to test whether an example sodium chlorite composition (greater than 99% purity, formulated for intravenous administration) affects levels of pathogenic cytokines thought to cause persistence of COVID-19 disease activity. Baseline values of certain markers in COVID-19 patients (e.g., as discussed herein) observed in in vitro studies revealed levels from 5 to up to 700 times above normal range. The sample size of 6 subjects per treatment group was selected to provide at least 80% power at a one-sided type I error rate of 0.10 for pairwise dose comparison to placebo to detect a coefficient of variation (CV) of up to 0.75.

An example sodium chlorite composition (greater than 99% purity, formulated for intravenous administration) can be administered in a single site, dose ranging, randomized, placebo-controlled, double-blind clinical trial with 2 dose cohorts:
Cohort 1: are administered the example sodium chlorite composition (2 mg/kg/dose) or saline placebo.
Cohort 2: are administered the example sodium chlorite composition (3 mg/kg/dose) or saline placebo.

Patients receive the sodium chlorite composition, in a 1 hour intravenous infusion per day for 5 days with baseline. At days 6 and 14 blood specimens are collected for evaluation of COVID-19 associated factor levels affected by the administration of the sodium chlorite composition.

Outcome measures assessed in this study can include recovery rate at day 14, duration of hospitalization, days with supplemental oxygen, incidence of mechanical ventilation, disease severity score at day 14, and mortality.

Safety, tolerability and inflammatory biomarkers (e.g., IL-1ra, IL-6, IL-10, G-CSF, IP-10 (CXCL10), IFN-y, MCP-1, MIP-1a, IL-2ra, IL-18, HGF, MCP-3, M-CSF, MIG (CXCL9), OPN, bactericidal/permeability-increasing protein (BPI), LPS binding protein (LBP), Calprotectin (S100A8/9), sCD14, and sCD163) are monitored throughout the study.

Sodium chlorite treatment is well tolerated and non-toxic to patients. Changes in baseline values for each of CXCL9, CXCL10, IL-1RA, IL-18, HGF, osteopontin (OPN), sCD163 and sCD14 etc. can parallel clinical reversal of symptoms of COVID-19.

### Example 8 - Microbiome Profiling of Covid-19 Patients

Total DNA was extracted from nasopharyngeal samples derived from 80 COVID-19 patients admitted to the Ochsner Health hospital in New Orleans during May 2020. Raw reads were quality filtered and aligned against a database of bacterial reads. The patients were then stratified by hospital outcome (e.g., alive/discharged patients vs. deceased, see FIG. 7, panels A and B respectively). Relative abundance of multiple taxonomic ranks were compared, between the two datasets.

FIG. 7, panels A and B illustrates the results for the taxonomic rank of Families. Interestingly, two families are statistically different between the two groups. Flavobacteriaceae were present at an average relative abundance at 5% in samples from discharged patients (panel A), although present at an average relative abundance of 18% in patients who died (panel B). In contrast, Propionibacteriaceae were present at an average relative abundance at 23% in samples from discharged patients (panel A), although present at an average relative abundance of 10% in patients who died (panel B).

These data reflect the microbiome of the nasopharyngeal passage and therefore are not directly reflective of the blood/gut microbiome; however, the differences between the two groups are intriguing and are consistent with a disruption of bacterial populations in the most severe cases.

In general, as seen herein, evidence for microbial translocation can be found in COVID-19 patients, and this can correlate with increased inflammation and disease outcome.

The preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the embodiments shown and described herein. Rather, the scope and spirit of present invention is embodied by the appended claims.

The scope of the present invention, therefore, is not intended to be limited to the example embodiments shown and described herein. Rather, the scope and spirit of present invention is embodied by the appended claims. In the claims, 35 U.S.C. §112(f) or 35 U.S.C. §112(6) is expressly defined as being invoked for a limitation in the claim only when the exact phrase “means for” or the exact phrase “step for” is recited at the beginning of such limitation in the claim; if such exact phrase is not used in a limitation in the claim, then 35 U.S.C. § 112 (f) or 35 U.S.C. §112(6) is not invoked.

What is claimed is:

1. A method of treating a subject having or at risk of developing acute respiratory distress syndrome (ARDS), the method comprising:
administering a chlorite composition to a subject having or at risk of developing ARDS, wherein the chlorite composition is administered in an amount effective to treat the subject.

2. The method of any one of claim 1, wherein chlorite is in the form of a pharmaceutically acceptable chlorite salt.

3. The method of claim 3, wherein the chlorite salt is sodium chlorite.

4. The method of any one of claims 1 to 3, wherein the chlorite composition is administered intravenously.

5. The method of any one of claims 1 to 4, wherein the chlorite is administered at a dose of from about 1 to 5 mg/kg of chlorite per day.

6. The method of claim 5, wherein the chlorite is administered to the subject for at least a 1 hour period.

7. The method of claim 5 or 6, wherein the chlorite is administered every day for one week or more.

8. The method of any one of claims 1 to 7, wherein the subject has or is suspected of having a coronavirus infection.

9. The method of claim 8, wherein the coronavirus is SARS-CoV, MERS-CoV, or SARS-CoV-2.

10. The method of claim 9, wherein the coronavirus is SARS-CoV-2.

11. The method of any one of claims 1 to 10, wherein the subject has or is suspected of having COVID-19.

12. The method of any one of claims 1 to 11, wherein the chlorite composition comprises a purified sodium chlorite, wherein the purified sodium chlorite is at least 98% pure.

13. The method of claim 12, wherein the composition is a liquid further comprising a pH adjusting agent.

14. The method of claim 13, wherein the pH adjusting agent is a phosphate buffer.

15. A method of treating a subject having or suspected of having a coronavirus infection, the method comprising:
administering a chlorite composition to a subject having or suspected of having a coronavirus infection, wherein the chlorite composition is administered in an amount effective to treat the subj ect.

16. The method of claim 15, wherein the coronavirus is HCoV-299E, HCoV-OC43, SARS-CoV, HCoV-NL63, HKU1, MERS-CoV, or SARS-CoV-2.

17. The method of claim 16, wherein the coronavirus is SARS-CoV-2.

18. The method of any one of claims 15 to 17, wherein the subject has or is at risk of developing acute respiratory distress syndrome (ARDS).

19. A method of regulating macrophage activity in a subject having or at risk of developing an acute respiratory distress syndrome (ARDS), comprising:
contacting a macrophage of a subject having or at risk of developing an acute respiratory distress syndrome (ARDS), with a composition comprising an amount of a chlorite composition effective to regulate one or more functional properties of the macrophage.

20. The method of claim 19, wherein the one or more functional properties comprises one or more of macrophage activation, a thrombotic process, a complement activation process, microbial translocation, a non-phagocytic phenotype, an inflammatory factor, and a monocytic migration process into a diseased tissue.

21. The method of claim 20, wherein the functional property is inappropriate macrophage activation, and the contacting results in the regulation of the macrophage activation.

22. The method of claim 20, wherein the functional property is an inflammatory factor, and the contacting decreases the level of the inflammatory factor activity as measured in the blood.

23. The method of claim 22, wherein the inflammatory factor is interleukin-18 (IL-18), C-X-C motif chemokine ligand 9 (CXCL9), C-X-C motif chemokine ligand 10 (CXCL10), interleukin-1 receptor antagonist (IL1Ra), osteopontin (OPN), or a combination thereof.

23a. The method of claim 19, wherein the functional property is a soluble plasma factor associated with one or more of cellular activation, inflammation, and infiltration of bacteria, and the contacting decreases the level of the soluble plasma factor activity as measured in the blood.

23b. The method of claim 23a, wherein the soluble plasma factor is C-X-C motif chemokine ligand 9 (CXCL9), C-X-C motif chemokine ligand 10 (CXCL10), interleukin-1 receptor antagonist (IL1Ra), hepatocyte growth factor (HGF), sCD163, osteopontin (OPN), or a combination thereof.

23c. The method of claim 19, wherein the functional property is a nuclear factor- κB (NF-κB), and the contacting decreases the NF-κB activity as measured in the blood.

24. The method of any one of claims 20 to 23, wherein the subject has or is suspected of having a coronavirus infection.

25. The method of claim 24, wherein the coronavirus is SARS-CoV, MERS-CoV, or SARS-CoV-2.

26. The method of claim 25, wherein the coronavirus is SARS-CoV-2.

27. The method of any one of claims 19 to 26, wherein chlorite is in the form of a pharmaceutically acceptable chlorite salt.

28. The method of claim 27, wherein the chlorite salt is sodium chlorite.

29. The method of any one of claims 20 to 28, wherein the chlorite composition comprises a purified sodium chlorite, wherein the purified sodium chlorite is at least 98% pure.

30. The method of claim 29, wherein the composition is in the form of a liquid further comprising a pH adjusting agent.

31. The method of claim 30, wherein the pH adjusting agent is a phosphate buffer.

32. The method of any one of claims 19 to 31, wherein the macrophage is in vitro.

33. The method of any one of claims 19 to 31, wherein the macrophage is in vivo.

34. The method of claim 33, wherein the contacting comprises administering an effective amount of the chlorite composition to an individual.

35. The method of claim 34, wherein the chlorite composition is administered intravenously.

36. The method of any one of claims 33 to 35, wherein the individual has been diagnosed with COVID-19.

37. A method of determining inhibition of an acute respiratory distress syndrome (ARDS)-induced macrophage activity by a chlorite agent, comprising:
contacting a macrophage of a subject having ARDS with a chlorite agent; and
assessing a level of one or more inflammatory factors selected from interleukin-18 (IL-18), C-X-C motif chemokine ligand 9 (CXCL9), C-X-C motif chemokine ligand 10 (CXCL10), osteopontin (OPN), and interleukin-1 receptor antagonist (IL1Ra) after said contacting,
wherein a decrease in the level of the inflammatory factor after said contacting indicates that the chlorite agent is an inhibitor of the ARDS-induced macrophage activity.

38. The method of claim 37, wherein the method further comprises:
measuring the level of the one or more inflammatory factors prior to said contacting.

39. The method of claim 37 or 38, wherein said assessing is at least one day after said contacting.

40. The method of any one of claims 37 to 39, wherein the inflammatory factor is C-X-C motif chemokine ligand 9 (CXCL9).

41. The method of any one of claims 37 to 39, wherein the inflammatory factor is interleukin-1 receptor antagonist (IL1Ra).

42. The method of any one of claims 37 to 39, wherein the inflammatory factor is C-X-C motif chemokine ligand 10 (CXCL10).

43. The method of any one of claims 37 to 39, wherein the inflammatory factor is osteopontin (OPN).
